(19) United States
(12) Patent Application Publication
(10) Pub. No.: US 2025/0009799 A1
(43) Pub. Date: Jan. 9, 2025

(54) DCAF4L2-SPECIFIC T-CELL RECEPTORS

(71) Applicant: AMGEN INC., Thousand Oaks, CA (US)

(72) Inventors: Sungeun KIM, San Mateo, CA (US); Yan ZHENG, Redwood City, CA (US); Kristin V. TARBELL, Milbrae, CA (US); Dhanashi S. BAGAL, Redwood City, CA (US); Sheree JOHNSTONE, San Mateo, CA (US)

(73) Assignee: AMGEN INC., Thousand Oaks, CA (US)

(21) Appl. No.: 18/709,897

(22) PCT Filed: Dec. 15, 2022
(86) PCT No.: PCT/US2022/052925

§ 371 Date: May 14, 2024

Related U.S. Application Data
(60) Provisional application No. 63/292,022, filed on Dec. 21, 2021.

(51) Int. Cl. A61K 35/17; A61K 39/00; C07K 14/54; C07K 14/725; C12N 5/0783; C12N 15/86

(52) U.S. Cl. CPC A61K 35/17; A61K 39/4611; A61K 39/4632; A61K 39/4635; C07K 14/5434; C07K 14/7051; C12N 5/0636; C12N 15/86; C12N 2510/00; C12N 2740/15043

(57) ABSTRACT

Provided herein are T-cell receptors (TCRs) that when expressed recombinantly on the surface of a T cell are able to recognize the DCAF4L2-derived peptide ILQDGQFLV (SEQ ID NO:1) when presented by HLA-A*02:01 sufficiently to activate the recombinant T cell. Importantly, exemplary TCRs provided herein were thoroughly screened for lack of cross-reactivity with similar peptides that may be presented by normal cells or tissue and for alloreactivity.

This application claims the benefit of U.S. Provisional Application No. 63/292,022, filed Dec. 21, 2021, which is hereby incorporated by reference in its entirety and for all purposes as if fully set forth herein.

### FIELD OF DISCLOSURE

The present invention relates to T-cell receptors that when expressed recombinantly on the surface of a T cell are able to recognize peptides sufficiently to activate the recombinant T cell.

### SEQUENCE LISTING

This application contains, as a separate part of the disclosure, a sequence list in computer-readable form (Filename: A-2912-WO01-SEC_Sequence_Listing.xml, created Sep. 13, 2022, which is 82 KB in size), and which is incorporated by reference in its entirety.

### BACKGROUND

Adoptive T cell therapies provide tremendous opportunities to treat cancer. Chimeric antigen receptor (CAR)-T cell therapy is an approved adoptive T cell therapy for hematological malignancy but has a limited range of targets due to its recognition to only cell surface antigens constituting ˜25% of the genome. Unlike CAR-T cells, TCR-T cells engineered to express the T cell receptors (TCR) specific to tumor antigens can exploit a broader range of targets for multiple cancer indications because TCR-T cells can recognize the peptide-MHC complexes (pMHC) derived from intracellular proteins constituting ˜75% of the genome. Intracellular proteins are processed and presented by major histocompatibility complex (MHC) as pMHC complexes.

Cancer-testis antigens (CTA) are attractive targets for cancer immunotherapy including TCR-T cell therapy due to their restricted expression in germ cells, aberrant reactivation in various cancers, and their immunogenic properties. Germ cells such as testis (immune-privileged sites) do not usually express HLA class I/II molecules, allowing them to evade attack from the immune system. DDB1 and CUL4 associated 4 like 2 (DCAF4L2) is a recently identified CTA and belongs to a large family of WD repeat-containing family member proteins that serve as substrate receptors of CUL4-DDB1 ubiquitin ligase complexes. A family of DDB1 and CUL4-associated factors (DCAFs) is shown to play roles in regulating DNA repair, cell proliferation, survival, and genomic integrity. A recent study suggests that DCAF4L2 may promote colorectal cancer cell invasion through mediating degradation of PPM1B, a negative regulator of NF-κB (Wang et al., Am J Transl Res. 2016, 8(2), 405). However, specific roles of DCAF family members including DCAF4L2 in cancer development remain largely unknown.

While TCR-T cells are shown to be very potent and sensitive modality for tumor specific peptide-MHC targets, a TCR can recognize multiple peptides. DNA rearrangement required for TCR formation generates a certain number of T cells that recognize self-antigens. During early T cell development, self-reactive T cells are negatively selected and eliminated in the medulla of the thymus through a promiscuous expression of a wide range of self-antigens in medullary thymic epithelial cells. This negative selection in the thymus functions as the major mechanism of central tolerance and shapes the T cell repertoire to avoid autoimmunity. TCRs that are engineered to increase their affinity for certain pMHC or to introduce cross-reactivity to multiple pMHC do not have the benefit of the negative selection that occurs in the thymus. It is noteworthy that affinity-enhanced MAGE-A3 TCR-T cells led to fatal toxicity due to cross-reactivity to Titin expressed in cardiac muscles (Cameron et al., Sci Transl Med. 2013, 5(197)).

### BRIEF SUMMARY OF THE INVENTION

Identification of TCR sequences recognizing tumor-specific antigens has been shown to be very challenging in the field particularly due to rarity of tumor-specific T cells in patient blood, difficulty in expanding a very small number of tumor-specific T cell clones ex vivo, and potential exhaustion or suppression of tumor-specific T cells in tumor infiltrating lymphocytes (TILs). Despite these challenges, provided herein are TCR sequences specific to DCAF4L2-MHC (ILQDGQFLV/HLA-A*02:01) identified by using healthy donor blood and an ex vivo stimulation method. As demonstrated in the Examples herein, the exemplary TCR-T cells recognizing the tumor-specific DCAF4L2 can be highly potent therapeutics for the treatment of DCAF4L2 HLA-A*02:01 tumors by exerting cytotoxicity and producing cytokines. These TCR-T cell therapies will be a significant treatment option for hepatocellular carcinoma (HCC)

TCR-T cells are the most potent and sensitive modality in vitro for pMHC targets. The TCR-T cells provided herein display high potency against even very low target-expressing cells. This high potency of TCR-T cells comes from the complex of the transduced TCR and endogenous CD3 subunits. In addition, to enhance in vivo efficacy, exemplary TCR-T cells comprise an activation-dependent IL12 payload that is incorporated into a TCR-T construct where IL12 expression is regulated by TCR activation under a composite promoter containing six NFAT (nuclear factor of activated T cells) response elements linked to a minimal IL-2 promoter. Therefore, when TCR-T-IL12 cells encounter tumor antigens, the IL12 is produced. We previously demonstrated that adoptive T cell therapy with IL12 payload enhanced the efficacy in preclinical mouse models in vivo and therefore could decrease potential clinical dose by 10-100×.

In a first aspect, the present invention is an expression vector comprising a nucleic acid sequence encoding a T-cell receptor (TCR) alpha chain and a TCR beta chain, wherein the TCR alpha chain and TCR beta chain are selected from the group consisting of a TCR alpha chain comprising an amino acid sequence set forth in SEQ ID NO:12 and a TCR beta chain comprising an amino acid sequence set forth in SEQ ID NO:23; a TCR alpha chain comprising an amino acid sequence set forth in SEQ ID NO:13 and a TCR beta chain comprising an amino acid sequence set forth in SEQ ID NO:24; a TCR alpha chain comprising an amino acid sequence set forth in SEQ ID NO:14 and a TCR beta chain comprising an amino acid sequence set forth in SEQ ID NO:25; a TCR alpha chain comprising an amino acid sequence set forth in SEQ ID NO:15 and a TCR beta chain comprising an amino acid sequence set forth in SEQ ID NO:26; a TCR alpha chain comprising an amino acid sequence set forth in SEQ ID NO:16 and a TCR beta chain comprising an amino acid sequence set forth in SEQ ID NO:27; a TCR alpha chain comprising an amino acid sequence set forth in SEQ ID NO:17 and a TCR beta chain comprising an amino acid sequence set forth in SEQ ID NO:28; a TCR alpha chain comprising an amino acid sequence set forth in SEQ ID NO:18 and a TCR beta chain comprising an amino acid sequence set forth in SEQ ID NO:29; a TCR alpha chain comprising an amino acid sequence set forth in SEQ ID NO:19 and a TCR beta chain comprising an amino acid sequence set forth in SEQ ID NO:30; a TCR alpha chain comprising an amino acid sequence set forth in SEQ ID NO:20 and a TCR beta chain comprising an amino acid sequence set forth in SEQ ID NO:31; a TCR alpha chain comprising an amino acid sequence set forth in SEQ ID NO:21 and a TCR beta chain comprising an amino acid sequence set forth in SEQ ID NO:32; and a TCR alpha chain comprising an amino acid sequence set forth in SEQ ID NO:22 and a TCR beta chain comprising an amino acid sequence set forth in SEQ ID NO:33.

Any expression vector of the first aspect may further comprising a nucleic acid encoding interleukin-12 (IL-12) or a functional variant thereof and may be a viral vector such as a retroviral or lentiviral vector.

In certain embodiments of the first aspect, the expression vector encodes a TCR alpha chain having a CDR3 region amino acid sequence as set forth in SEQ ID NO:12 and the TCR beta chain a CDR3 region amino acid sequence as set forth in SEQ ID NO:23. In preferred embodiments, the mature TCR alpha chain comprises an amino acid sequence set forth in SEQ ID NO:34 and the mature TCR beta chain comprises an amino acid sequence set forth in SEQ ID NO:45. The expression vector may encode the full-length TCR alpha chain comprising the amino acid sequence set forth in SEQ ID NO:56 and the full-length TCR beta chain comprising the amino acid sequence set forth in SEQ ID NO:67.

In certain embodiments of the first aspect, the expression vector encodes a TCR alpha chain having a CDR3 region amino acid sequence as set forth in SEQ ID NO:13 and the TCR beta chain a CDR3 region amino acid sequence as set forth in SEQ ID NO:24. In preferred embodiments, the mature TCR alpha chain comprises an amino acid sequence set forth in SEQ ID NO:35 and the mature TCR beta chain comprises an amino acid sequence set forth in SEQ ID NO:46. The expression vector may encode the full-length TCR alpha chain comprising the amino acid sequence set forth in SEQ ID NO:57 and the full-length TCR beta chain comprising the amino acid sequence set forth in SEQ ID NO:68.

In certain embodiments of the first aspect, the expression vector encodes a TCR alpha chain having a CDR3 region amino acid sequence as set forth in SEQ ID NO:14 and the TCR beta chain a CDR3 region amino acid sequence as set forth in SEQ ID NO:25. In preferred embodiments, the mature TCR alpha chain comprises an amino acid sequence set forth in SEQ ID NO:36 and the mature TCR beta chain comprises an amino acid sequence set forth in SEQ ID NO:47. The expression vector may encode the full-length TCR alpha chain comprising the amino acid sequence set forth in SEQ ID NO:58 and the full-length TCR beta chain comprising the amino acid sequence set forth in SEQ ID NO:69.

In certain embodiments of the first aspect, the expression vector encodes a TCR alpha chain having a CDR3 region amino acid sequence as set forth in SEQ ID NO:15 and the TCR beta chain a CDR3 region amino acid sequence as set forth in SEQ ID NO:26. In preferred embodiments, the mature TCR alpha chain comprises an amino acid sequence set forth in SEQ ID NO:37 and the mature TCR beta chain comprises an amino acid sequence set forth in SEQ ID NO:48. The expression vector may encode the full-length TCR alpha chain comprising the amino acid sequence set forth in SEQ ID NO:59 and the full-length TCR beta chain comprising the amino acid sequence set forth in SEQ ID NO:70.

In certain embodiments of the first aspect, the expression vector encodes a TCR alpha chain having a CDR3 region amino acid sequence as set forth in SEQ ID NO:16 and the TCR beta chain a CDR3 region amino acid sequence as set forth in SEQ ID NO:27. In preferred embodiments, the mature TCR alpha chain comprises an amino acid sequence set forth in SEQ ID NO:38 and the mature TCR beta chain comprises an amino acid sequence set forth in SEQ ID NO:49. The expression vector may encode the full-length TCR alpha chain comprising the amino acid sequence set forth in SEQ ID NO:60 and the full-length TCR beta chain comprising the amino acid sequence set forth in SEQ ID NO:71.

In certain embodiments of the first aspect, the expression vector encodes a TCR alpha chain having a CDR3 region amino acid sequence as set forth in SEQ ID NO:19 and the TCR beta chain a CDR3 region amino acid sequence as set forth in SEQ ID NO:28. In preferred embodiments, the mature TCR alpha chain comprises an amino acid sequence set forth in SEQ ID NO:39 and the mature TCR beta chain comprises an amino acid sequence set forth in SEQ ID NO:50. The expression vector may encode the full-length TCR alpha chain comprising the amino acid sequence set forth in SEQ ID NO:61 and the full-length TCR beta chain comprising the amino acid sequence set forth in SEQ ID NO:72.

In certain embodiments of the first aspect, the expression vector encodes a TCR alpha chain having a CDR3 region amino acid sequence as set forth in SEQ ID NO:18 and the TCR beta chain a CDR3 region amino acid sequence as set forth in SEQ ID NO:29. In preferred embodiments, the mature TCR alpha chain comprises an amino acid sequence set forth in SEQ ID NO:40 and the mature TCR beta chain comprises an amino acid sequence set forth in SEQ ID NO:51. The expression vector may encode the full-length TCR alpha chain comprising the amino acid sequence set forth in SEQ ID NO:62 and the full-length TCR beta chain comprising the amino acid sequence set forth in SEQ ID NO:73.

In certain embodiments of the first aspect, the expression vector encodes a TCR alpha chain having a CDR3 region amino acid sequence as set forth in SEQ ID NO:19 and the TCR beta chain a CDR3 region amino acid sequence as set forth in SEQ ID NO:30. In preferred embodiments, the mature TCR alpha chain comprises an amino acid sequence set forth in SEQ ID NO:41 and the mature TCR beta chain comprises an amino acid sequence set forth in SEQ ID NO:52. The expression vector may encode the full-length TCR alpha chain comprising the amino acid sequence set forth in SEQ ID NO:63 and the full-length TCR beta chain comprising the amino acid sequence set forth in SEQ ID NO:74.

In certain embodiments of the first aspect, the expression vector encodes a TCR alpha chain having a CDR3 region amino acid sequence as set forth in SEQ ID NO:20 and the TCR beta chain a CDR3 region amino acid sequence as set forth in SEQ ID NO:31. In preferred embodiments, the mature TCR alpha chain comprises an amino acid sequence set forth in SEQ ID NO:42 and the mature TCR beta chain comprises an amino acid sequence set forth in SEQ ID NO:53. The expression vector may encode the full-length TCR alpha chain comprising the amino acid sequence set forth in SEQ ID NO:64 and the full-length TCR beta chain comprising the amino acid sequence set forth in SEQ ID NO:75.

In certain embodiments of the first aspect, the expression vector encodes a TCR alpha chain having a CDR3 region amino acid sequence as set forth in SEQ ID NO:21 and the TCR beta chain a CDR3 region amino acid sequence as set forth in SEQ ID NO:32. In preferred embodiments, the mature TCR alpha chain comprises an amino acid sequence set forth in SEQ ID NO:43 and the mature TCR beta chain comprises an amino acid sequence set forth in SEQ ID NO:54. The expression vector may encode the full-length TCR alpha chain comprising the amino acid sequence set forth in SEQ ID NO:65 and the full-length TCR beta chain comprising the amino acid sequence set forth in SEQ ID NO:76.

In certain embodiments of the first aspect, the expression vector encodes a TCR alpha chain having a CDR3 region amino acid sequence as set forth in SEQ ID NO:22 and the TCR beta chain a CDR3 region amino acid sequence as set forth in SEQ ID NO:33. In preferred embodiments, the mature TCR alpha chain comprises an amino acid sequence set forth in SEQ ID NO:44 and the mature TCR beta chain comprises an amino acid sequence set forth in SEQ ID NO:55. The expression vector may encode the full-length TCR alpha chain comprising the amino acid sequence set forth in SEQ ID NO:66 and the full-length TCR beta chain comprising the amino acid sequence set forth in SEQ ID NO:77.

In a second aspect, is a cell expressing a recombinant T-cell receptor (TCR), said TCR comprising a TCR alpha chain CDR3 region comprising an amino acid sequence set forth in SEQ ID NO:12 and a TCR beta chain CDR3 region comprising an amino acid sequence set forth in SEQ ID NO:23; a TCR alpha chain CDR3 region comprising an amino acid sequence set forth in SEQ ID NO:13 and a TCR beta chain CDR3 region comprising an amino acid sequence set forth in SEQ ID NO:24; a TCR alpha chain CDR3 region comprising an amino acid sequence set forth in SEQ ID NO:14 and a TCR beta chain CDR3 region comprising an amino acid sequence set forth in SEQ ID NO:25; a TCR alpha chain CDR3 region comprising an amino acid sequence set forth in SEQ ID NO:15 and a TCR beta chain CDR3 region comprising an amino acid sequence set forth in SEQ ID NO:26; a TCR alpha chain CDR3 region comprising an amino acid sequence set forth in SEQ ID NO:16 and a TCR beta chain CDR3 region comprising an amino acid sequence set forth in SEQ ID NO:27; a TCR alpha chain CDR3 region comprising an amino acid sequence set forth in SEQ ID NO:17 and a TCR beta chain CDR3 region comprising an amino acid sequence set forth in SEQ ID NO:28; a TCR alpha chain CDR3 region comprising an amino acid sequence set forth in SEQ ID NO:18 and a TCR beta chain CDR3 region comprising an amino acid sequence set forth in SEQ ID NO:29; a TCR alpha chain CDR3 region comprising an amino acid sequence set forth in SEQ ID NO:19 and a TCR beta chain CDR3 region comprising an amino acid sequence set forth in SEQ ID NO:30; a TCR alpha chain CDR3 region comprising an amino acid sequence set forth in SEQ ID NO:20 and a TCR beta chain CDR3 region comprising an amino acid sequence set forth in SEQ ID NO:31; a TCR alpha chain CDR3 region comprising an amino acid sequence set forth in SEQ ID NO:21 and a TCR beta chain CDR3 region comprising an amino acid sequence set forth in SEQ ID NO:32; or a TCR alpha chain CDR3 region comprising an amino acid sequence set forth in SEQ ID NO:22 and a TCR beta chain CDR3 region comprising an amino acid sequence set forth in SEQ ID NO:33.

In preferred embodiments of the second aspect, the cell recombinantly expresses a TCR comprising a TCR alpha chain comprising an amino acid sequence set forth in SEQ ID NO:34 and a TCR beta chain comprising an amino acid sequence set forth in SEQ ID NO:45; a TCR alpha chain comprising an amino acid sequence set forth in SEQ ID NO:35 and a TCR beta chain comprising an amino acid sequence set forth in SEQ ID NO:46; a TCR alpha chain comprising an amino acid sequence set forth in SEQ ID NO:36 and a TCR beta chain comprising an amino acid sequence set forth in SEQ ID NO:47; a TCR alpha chain comprising an amino acid sequence set forth in SEQ ID NO:37 and a TCR beta chain comprising an amino acid sequence set forth in SEQ ID NO:48; a TCR alpha chain comprising an amino acid sequence set forth in SEQ ID NO:38 and a TCR beta chain comprising an amino acid sequence set forth in SEQ ID NO:49; a TCR alpha chain comprising an amino acid sequence set forth in SEQ ID NO:39 and a TCR beta chain comprising an amino acid sequence set forth in SEQ ID NO:50; a TCR alpha chain comprising an amino acid sequence set forth in SEQ ID NO:40 and a TCR beta chain comprising an amino acid sequence set forth in SEQ ID NO:51; a TCR alpha chain comprising an amino acid sequence set forth in SEQ ID NO:41 and a TCR beta chain comprising an amino acid sequence set forth in SEQ ID NO:52; a TCR alpha chain comprising an amino acid sequence set forth in SEQ ID NO:42 and a TCR beta chain comprising an amino acid sequence set forth in SEQ ID NO:53; a TCR alpha chain comprising an amino acid sequence set forth in SEQ ID NO:43 and a TCR beta chain comprising an amino acid sequence set forth in SEQ ID NO:54; or a TCR alpha chain comprising an amino acid sequence set forth in SEQ ID NO:44 and a TCR beta chain comprising an amino acid sequence set forth in SEQ ID NO:55.

The cell of the second aspect further may express a recombinant IL-12 or functional variant thereof. In certain embodiments of the second aspect, the cell comprises one or more expression vectors of the first aspect. The cell may be a T cell and, when the TCR binds the peptide of SEQ ID NO:1 in the context of HLA-A*02:01, the binding leads to activation of IFNγ, TNFα, or granzyme B production by the cell.

In a third aspect of the invention, a pharmaceutical composition comprises a therapeutically effect amount of a cell of the second aspect or an expression vector of the first aspect.

In a fourth aspect, the invention provides a method of making a cell of the second aspect or a pharmaceutical composition of the third aspect, comprising introducing into a cell an expression vector comprising a nucleic acid sequence encoding a TCR alpha chain and a TCR beta chain, wherein the TCR alpha chain and TCR beta chain are selected from the group consisting of a TCR alpha chain comprising a CDR3 region having an amino acid sequence set forth in SEQ ID NO:12 and a TCR beta chain comprising a CDR3 region having an amino acid sequence set forth in SEQ ID NO:23; a TCR alpha chain comprising a CDR3 region having an amino acid sequence set forth in SEQ ID NO:13 and a TCR beta chain comprising a CDR3 region having an amino acid sequence set forth in SEQ ID NO:24; a TCR alpha chain comprising a CDR3 region having an amino acid sequence set forth in SEQ ID NO:14 and a TCR beta chain comprising a CDR3 region having an amino acid sequence set forth in SEQ ID NO:25; a TCR alpha chain comprising a CDR3 region having an amino acid sequence set forth in SEQ ID NO:15 and a TCR beta chain comprising a CDR3 region having an amino acid sequence set forth in SEQ ID NO:26; a TCR alpha chain comprising a CDR3 region having an amino acid sequence set forth in SEQ ID NO:16 and a TCR beta chain comprising a CDR3 region having an amino acid sequence set forth in SEQ ID NO:27; a TCR alpha chain comprising a CDR3 region having an amino acid sequence set forth in SEQ ID NO:17 and a TCR beta chain comprising a CDR3 region having an amino acid sequence set forth in SEQ ID NO:28; a TCR alpha chain comprising a CDR3 region having an amino acid sequence set forth in SEQ ID NO:18 and a TCR beta chain comprising a CDR3 region having an amino acid sequence set forth in SEQ ID NO:29; a TCR alpha chain comprising a CDR3 region having an amino acid sequence set forth in SEQ ID NO:19 and a TCR beta chain comprising a CDR3 region having an amino acid sequence set forth in SEQ ID NO:30; a TCR alpha chain comprising a CDR3 region having an amino acid sequence set forth in SEQ ID NO:20 and a TCR beta chain comprising a CDR3 region having an amino acid sequence set forth in SEQ ID NO:31; a TCR alpha chain comprising a CDR3 region having an amino acid sequence set forth in SEQ ID NO:21 and a TCR beta chain comprising a CDR3 region having an amino acid sequence set forth in SEQ ID NO:32; or a TCR alpha chain comprising a CDR3 region having an amino acid sequence set forth in SEQ ID NO:22 and a TCR beta chain comprising a CDR3 region having an amino acid sequence set forth in SEQ ID NO:33.

In preferred embodiments of the fourth aspect, the TCR alpha chain and TCR beta chain are selected from the group consisting of a TCR alpha chain comprising an amino acid sequence set forth in SEQ ID NO:34 and a TCR beta chain comprising an amino acid sequence set forth in SEQ ID NO:45; a TCR alpha chain comprising an amino acid sequence set forth in SEQ ID NO:35 and a TCR beta chain comprising an amino acid sequence set forth in SEQ ID NO:46; a TCR alpha chain comprising an amino acid sequence set forth in SEQ ID NO:36 and a TCR beta chain comprising an amino acid sequence set forth in SEQ ID NO:47; a TCR alpha chain comprising an amino acid sequence set forth in SEQ ID NO:37 and a TCR beta chain comprising an amino acid sequence set forth in SEQ ID NO:48; a TCR alpha chain comprising an amino acid sequence set forth in SEQ ID NO:38 and a TCR beta chain comprising an amino acid sequence set forth in SEQ ID NO:49; a TCR alpha chain comprising an amino acid sequence set forth in SEQ ID NO:39 and a TCR beta chain comprising an amino acid sequence set forth in SEQ ID NO:50; a TCR alpha chain comprising an amino acid sequence set forth in SEQ ID NO:40 and a TCR beta chain comprising an amino acid sequence set forth in SEQ ID NO:51; a TCR alpha chain comprising an amino acid sequence set forth in SEQ ID NO:41 and a TCR beta chain comprising an amino acid sequence set forth in SEQ ID NO:52; a TCR alpha chain comprising an amino acid sequence set forth in SEQ ID NO:42 and a TCR beta chain comprising an amino acid sequence set forth in SEQ ID NO:53; a TCR alpha chain comprising an amino acid sequence set forth in SEQ ID NO:43 and a TCR beta chain comprising an amino acid sequence set forth in SEQ ID NO:54; and a TCR alpha chain comprising an amino acid sequence set forth in SEQ ID NO:44 and a TCR beta chain comprising an amino acid sequence set forth in SEQ ID NO:55.

In certain embodiments of the fourth aspect, a nucleic acid sequence encoding IL-12 or a functional variant thereof is also introduced into the cell and may be on an expression vector encoding the alpha chain and/or beta chain, or may encoded on a separate vector. The cell made by a method of the fourth aspect may be a primary T cell isolated from a cancer patient.

In a fifth aspect, the invention provides methods of treating a DCAF4L2 expressing cancer, said method comprising administering to a cancer patient a therapeutically effective amount of a cell of the second aspect, a pharmaceutical composition of the third aspect, or of a cell made by the method of the fourth aspect. In certain embodiments of the fifth aspect, the patient is tested prior to administration to determine the presence of a cancer expressing DCAF4L2. The test may detect a DCAF4L2-encoding nucleic acid, a DCAF4L2 protein or a DCAF4L2-derived peptide. In preferred embodiments, the patient is identified as carrying the HLA-A*02:01 allele.

### BRIEF DESCRIPTION OF THE DRAWINGS

FIG. 1. DCAF4L2 is overexpressed in hepatocellular carcinoma. A) TCGA transcriptome analysis reveals elevated DCAF4L2 expression in HCC. Numbers denote number of tumor samples per cancer type. B) Mass spectrometry (MS) analysis of normal tissues and primary tumor samples. Three subgroups of primary normal tissues based on risk levels (high, medium, and low) are shown. MS signal intensity correlates to presence of HLA-A*02:01 bound ILQDGQFLV peptide. Detection frequency denotes the proportion of tissue samples for which ILQGDQFLV-pMHC (HLA-A*02:01) was detected.

FIG. 2. Identification of DCAF4L2 pMHC-specific TCRs from healthy human PBMCs. (A) A schematic illustrates the procedure of identifying DCAF4L2 pMHC-specific TCRs from rare T cell clones isolated from healthy HLA-A*02:01+ donor PBMCs. (B) Flow cytometric identification of DCAF4L2 pMHC-specific T cells by pMHC dextramers (Dex) labelled with two fluorochromes (PE and APC) following multiple rounds of enrichment through stimulation with DCAF4L2 peptide-loaded autologous antigen presenting cells. A representative positive donor A showed the enriched DCAF4L2 pMHC-specific T cells after multiple ex vivo stimulation, whereas a negative donor B did not have Dex+ T cells. (C) IFNγ ELISPOT analysis of sorted CD8+Dex+ T cells that were stimulated with T2 cells pulsed with a DCAF4L2 peptide or an irrelevant AFP peptide as a negative control.

FIG. 3. Selection of top TCRs through in vitro potency evaluation assays. TCRs were transduced into TCRαKO/TCRβKO/CD8a/NFAT-luciferase reporter Jurkat cell lines and incubated with DCAF4L2 peptide-pulsed T2 cells for 24 hours. TCR potency was evaluated by quantifying NFAT-induced (TCR activation-dependent) luciferase expression. T cells transduced with the DMF5 TCR (MART-1 peptide-HLA-A*02:01) were included as negative controls. TCR potency was ranked by fold enhancement in luciferase expression of TCR-Ts following exposure to 10⁻⁵M and 10⁻⁷M peptide pulsed or non-peptide loaded T2 cells.

FIG. 4. Potency evaluation of top 11 TCRs in human primary pan T cells expressing individual TCRs (TCR-Ts). TCR-T cells were evaluated in T2 TDCC assays with E:T titration and peptide titration. Key characteristics including median GFP, median dextramer frequencies, median EC50 and median EC90 were determined for primary TCR-T cells generated from 3 different donors.

FIG. 5. Potency validation of top 5 TCR-Ts in DCAF4L2+ HLA-A*02:01+ cancer cell lines. (A) Representative TDCC activities of top DCAF4L2 TCR-Ts against KMM-1.Luc cancer cell line. (B) DCAF4L2 KO in KMM-1.Luc cancer cell line led to the loss of cytolytic activity of all 5 TCR-Ts.

FIG. 6. Schematic diagram of the TCR-T-IL12 lentiviral construct containing TCRα and TCRβ chains with a linker of furin cleavage site-SGSG-T2A under EF1α promoter, and IL12 payload under a composite promoter containing six NFAT (nuclear factor of activated T cells) response elements linked to a minimal IL-2 promoter.

FIG. 7. Potency validation of TCR2-IL-12 T cells. TCR-2-IL12 cells displayed potent cytolytic activities against DCAF4L2 expressing cancer cell lines such as KMM-1 (A), NCI-H2023 (B), and HLA-A*02:01-overexpressing AU565 (C). Negligible TCR-T cytolytic activity was observed against DCAF4L2 negative cancer cell lines such as T98C (D) and UACC257 (E). All the specific killing activities (%) (A-E) were calculated by normalizing cytolytic activity of TCR-T-IL12 by cytolytic activity of IL12-RFP T cells (NFAT.IL-12.RFP transduced T cells without transgenic TCR as a negative control).

FIG. 8. Identification of putative cross-reactive peptides for TCR2-IL 12, through testing of full panel of similar peptides using T2/peptide TDCC assay. (a) Cross-reactivity potency screen of 7 peptides for the TCR2-IL12 cells identified a single putative cross-reactive peptide arising from SH2D3A, exhibiting a potency gap of less than 10³-fold in EC50 between target peptide and putative peptide. (b) Typical cytolytic activity of TCR2-IL 12 against DCAF4L2 peptide loaded T2 cells compared to IL12-RFP negative control T cells.

FIG. 9. Evaluation of cross-reactivity of putative protein SH2D3A. Full-length protein SH2D3A or DCAF4L2 (as a positive control) was overexpressed in two DCAF4L2 negative/HLA-A*02:01+ cancer cell lines such as T98G (C) and UACC257 (D). The full-length protein overexpression in each cancer cell line compared to wild type cancer cell lines was confirmed by Western Blot (WB) (C and D). P-actin that is a housekeeping protein was used for a loading control in WB. TCR2-IL12 TCR-T cells displayed potent cytolytic activity against DCAF4L2-overexpressing T98G (A) or UACC257 (B) cancer cell lines as expected, while TCR2-T-IL12 T cells showed negligible cytolytic activity against SH2D3A-overexpressing T98G (A) or UACC257 (B), suggesting that this peptide is unlikely to be naturally processed and presented from the protein.

FIG. 10. Summary of human normal cell cytotoxicity assessment. No strong caspase 3/7 activation was observed when TCR2-IL12 T cells were co-cultured with any of the normal cell types, despite some low-level response with hBEpC and HGN cells. TCR2-IL12 T cells or IL12-RFP control T cells were co-cultured with the DCAF4L2+ HLA-A*02:01+ cancer cell line NCI-H2023 as a positive control (A), or HLA-A*02:01+ human primary or iPSC-derived cells as follows: B) RPTEC (renal proximal tubule epithelial cells); C) hTEpC (tracheal epithelial cells); D) hBEpC (bronchial epithelial cells); E) HDMEC (dermal microvascular endothelial cells); F) HCM (cardiomyocytes); G) HA (iPSC-derived astrocytes); H) NHEK (epidermal keratinocytes); I) HEP (hepatocytes); J) HGN (GABA neurons). Enzymatic activity of caspase 3/7, quantified by total green object integrated intensity, was kinetically monitored by IncuCyte® using a fluorogenic substrate. Total integrated intensity=pixel intensity of green fluorescent emission in green calibrated units (GCU)×object area in m²/image.

FIG. 11. Summary of human normal cell reactivity assessment. Granzyme B and cytokines were measured from the culture supernatant described in FIG. 10. No increases greater than or equal to 3-fold (compared to the IL12-RFP control T cells) in granzyme B, or TNFα production was observed when TCR2-IL12 T cells were co-cultured with any of the nine normal cell types tested. Increases in IFNγ production greater than 3-fold (but less than 10-fold) were observed with select cell types, including hTEpC, HCM, NHEK HEP, and HGN.

FIG. 12. Summary of alloreactivity assessment. Cytokine and granzyme B response following co-culture of TCR2-IL12 T cells with each of 34 BLCLs. U266B1+pep refers to HLA-A*02:01′ U266B1 cell line pulsed with 50 μM DCAF4L2 peptide ILQDGQFLV as a positive control. No increases greater than or equal to 3-fold in granzyme B, IFNγ or TNF α (compared to IL12-RFP control T cells) were observed. TCR2-IL12 cells demonstrated robust cytokine and granzyme B production in response to the positive control U266B1 cell line pulsed with DCAF4L2 peptide.

FIG. 13. Crystal structure and interaction identification of the DCAF4L2 pMHC/TCR2 complex. TCR2 in complex with DCAF4L2 pMHC. TCR2 α- and β-chains are shown in dark and light grey respectively. Peptide-MHC is shown in medium grey with the peptide at the TCR2/pMHC interface.

FIG. 14. Crystal Structure and interaction identification of the DCAF4L2 pMHC/TCR2 complex. Positions of the CDRs in relation to the DCAF4L2 peptide and the HLA are shown.

### DETAILED DESCRIPTION OF THE INVENTION

The section headings used herein are for organizational purposes only and are not to be construed as limiting the subject matter described. All references cited within the body of this specification are expressly incorporated by reference in their entirety.

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3rd ed., Cold Spring Harbor Laboratory Press, cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

Provided herein are T-cell receptor (TCR) alpha and beta chain pairs that bind the DCAF4L2 derived peptide ILQDGQFLV (SEQ ID NO:1) when presented by an HLA class I molecule, preferably HLA-A*02:01. “TCR alpha and beta chain pair” may also be referred to herein as “TCR,” “a TCR,” or “the TCR.” When expressed recombinantly in a cell, e.g., a T cell, the TCR binds to the DCAF4L2-HLA complex on a cell, e.g., a cancer cell, and such binding leads to activation of the recombinant cell. Activation of the T cell leads to the death or destruction of the cancer cell. Methods of determining T-cell activation are known in the art and provided with the Examples herein.

In preferred embodiments, the potency or cytolytic activity (cytotoxicity) of a recombinant cell of the present invention is defined by (1) 80-100% lysis of HLA-A*02:01 target cells loaded with peptide at ˜100 copies (˜10⁻⁸ M) per cell in a TDCC T2 loading assay or (2) 80-100% lysis of natural pMHC target-positive cancer cell lines.

Each TCR alpha and beta chain comprises variable and constant domains. Within the variable domain (Vα or Vβ) are three CDRs (complementarity determining regions): CDR1, CDR2, and CDR3. The various alpha and beta chains variable domains are distinguishable by their framework along with their CDR1, CDR2, and part of their CDR3 sequences. Table 1 provides amino acid sequences of TCR alpha chain and TCR beta chain CDR3, mature sequences, and with signal peptides.

TABLE 1
Amino acid sequences of TCR alpha and beta chain CDR3, mature sequence and sequences
with signal peptides.

| SEQ ID | | |
|---|---|---|
| NO: | Description | Sequence |
| 12 | TCR1 alpha | CVLKGGGNKLTF |
| | chain CDR3 | |
| 13 | TCR2 alpha | CAVQAWGGGSQGNLIF |
| | chain CDR3 | |
| 14 | TCR3 alpha | CVVNGINRGSTLGRLYF |
| | chain CDR3 | |
| 15 | TCR4 alpha | CVVSAYARLMF |
| | chain CDR3 | |
| 16 | TCR5 alpha | CGADELGIQGAQKLVF |
| | chain CDR3 | |
| 17 | TCR6 alpha | CVVIEGNYGGATNKLIF |
| | chain CDR3 | |
| 18 | TCR7 alpha | CAMREPYKYNNNDMRF |
| | chain CDR3 | |
| 19 | TCR8 alpha | CAYPLVNQGGKLIF |
| | chain CDR3 | |
| 20 | TCR9 alpha | CGALASGGYQKVTF |
| | chain CDR3 | |
| 21 | TCR10 alpha | CAVSNMDSSYKLIF |
| | chain CDR3 | |
| 22 | TCR11 alpha | CAVPTGNTPLVF |
| | chain CDR3 | |
| 23 | TCR1 beta chain | CASSYPTGAYYGYTF |
| | CDR3 | |
| 24 | TCR2 beta chain | CASSAGDRGYEQYF |
| | CDR3 | |
| 25 | TCR3 beta chain | CASSGGYWDQENTEAFF |
| | CDR3 | |
| 26 | TCR4 beta chain | CASSLGGQGGAGELFF |
| | CDR3 | |
| 27 | TCR5 beta chain | CASSKVAGAIETQYF |
| | CDR3 | |
| 28 | TCR6 beta chain | CASSKGIRYEQYF |
| | CDR3 | |
| 29 | TCR7 beta chain | CASSARQGTNEKLFF |
| | CDR3 | |
| 30 | TCR8 beta chain | CASSLVATWDEQFF |
| | CDR3 | |
| 31 | TCR9 beta chain | CASSPSGTPSGANVLTF |
| | CDR3 | |
| 32 | TCR10 beta | CSALPSGVDRLNTEAFF |
| | chain CDR3 | |
| 33 | TCR11 beta | CAATGASRPTEAFF |
| | chain CDR3 | |
| 34 | TCR1 alpha | AQSVTQLDSHVSVSEGTPVLLRCNYSSSYSPSLFWYVQHPNKGL |
| | chain mature | QLLLKYTSAATLVKGINGFEAEFKKSETSFHLTKPSAHMSDAAE |
| | | YFCVLKGGGNKLTFGTGTQLKVELNIQNPDPAVYQLRDSKSSD |
| | | KSVCLFTDFDSQTNVSQSKDSDVYITDKTVLDMRSMDFKSNSA |
| | | VAWSNKSDFACANAFNNSIIPEDTFFPSPESSCDVKLVEKSFETD |
| | | TNLNFQNLSVIGFRILLLKVAGFNLLMTLRLWSS |
| 35 | TCR2 alpha | EDQVTQSPEALRLQEGESSSLNCSYTVSGLRGLFWYRQDPGKG |
| | chain mature | PEFLFTLYSAGEEKEKERLKATLTKKESFLHITAPKPEDSATYLC |
| | | AVQAWGGGSQGNLIFGKGTKLSVKPNIQNPDPAVYQLRDSKSS |
| | | DKSVCLFTDFDSQTNVSQSKDSDVYITDKTVLDMRSMDFKSNS |
| | | AVAWSNKSDFACANAFNNSIIPEDTFFPSPESSCDVKLVEKSFET |
| | | DTNLNFQNLSVIGFRILLLKVAGFNLLMTLRLWSS |
| 36 | TCR3 alpha | RKEVEQDPGPFNVPEGATVAFNCTYSNSASQSFFWYRQDCRKE |
| | chain mature | PKLLMSVYSSGNEDGRFTAQLNRASQYISLLIRDSKLSDSATYL |
| | | CVVNGINRGSTLGRLYFGRGTQLTVWPDIQNPDPAVYQLRDSK |
| | | SSDKSVCLFTDFDSQTNVSQSKDSDVYITDKTVLDMRSMDFKS |
| | | NSAVAWSNKSDFACANAFNNSIIPEDTFFPSPESSCDVKLVEKSF |
| | | ETDTNLNFQNLSVIGFRILLLKVAGFNLLMTLRLWSS |
| 37 | TCR4 alpha | KNQVEQSPQSLIILEGKNCTLQCNYTVSPFSNLRWYKQDTGRGP |
| | chain mature | VSLTIMTFSENTKSNGRYTATLDADTKQSSLHITASQLSDSASYI |
| | | CVVSAYARLMFGDGTQLVVKPNIQNPDPAVYQLRDSKSSDKSV |
| | | CLFTDFDSQTNVSQSKDSDVYITDKTVLDMRSMDFKSNSAVAW |
| | | SNKSDFACANAFNNSIIPEDTFFPSPESSCDVKLVEKSFETDTNLN |
| | | FQNLSVIGFRILLLKVAGFNLLMTLRLWSS |
| 38 | TCR5 alpha | SQELEQSPQSLIVQEGKNLTINCTSSKTLYGLYWYKQKYGEGLI |
| | chain mature | FLMMLQKGGEEKSHEKITAKLDEKKQQSSLHITASQPSHAGIYL |
| | | CGADELGIQGAQKLVFGQGTRLTINPNIQNPDPAVYQLRDSKSS |
| | | DKSVCLFTDFDSQTNVSQSKDSDVYITDKTVLDMRSMDFKSNS |
| | | AVAWSNKSDFACANAFNNSIIPEDTFFPSPESSCDVKLVEKSFET |
| | | DTNLNFQNLSVIGFRILLLKVAGFNLLMTLRLWSS |
| 39 | TCR6 alpha | RKEVEQDPGPFNVPEGATVAFNCTYSNSASQSFFWYRQDCRKE |
| | chain mature | PKLLMSVYSSGNEDGRFTAQLNRASQYISLLIRDSKLSDSATYL |
| | | CVVIEGNYGGATNKLIFGTGTLLAVQPNIQNPDPAVYQLRDSKS |
| | | SDKSVCLFTDFDSQTNVSQSKDSDVYITDKTVLDMRSMDFKSN |
| | | SAVAWSNKSDFACANAFNNSIIPEDTFFPSPESSCDVKLVEKSFE |
| | | TDTNLNFQNLSVIGFRILLLKVAGFNLLMTLRLWSS |
| 40 | TCR7 alpha | AQKITQTQPGMFVQEKEAVTLDCTYDTSDQSYGLFWYKQPSSG |
| | chain mature | EMIFLIYQGSYDEQNATEGRYSLNFQKARKSANLVISASQLGDS |
| | | AMYFCAMREPYKYNNNDMRFGAGTRLTVKPNIQNPDPAVYQL |
| | | RDSKSSDKSVCLFTDFDSQTNVSQSKDSDVYITDKTVLDMRSM |
| | | DFKSNSAVAWSNKSDFACANAFNNSIIPEDTFFPSPESSCDVKLV |
| | | EKSFETDTNLNFQNLSVIGFRILLLKVAGFNLLMTLRLWSS |
| 41 | TCR8 alpha | AQTVTQSQPEMSVQEAETVTLSCTYDTSESDYYLFWYKQPPSR |
| | chain mature | QMILVIRQEAYKQQNATENRFSVNFQKAAKSFSLKISDSQLGDA |
| | | AMYFCAYPLVNQGGKLIFGQGTELSVKPNIQNPDPAVYQLRDS |
| | | KSSDKSVCLFTDFDSQTNVSQSKDSDVYITDKTVLDMRSMDFK |
| | | SNSAVAWSNKSDFACANAFNNSIIPEDTFFPSPESSCDVKLVEKS |
| | | FETDTNLNFQNLSVIGFRILLLKVAGFNLLMTLRLWSS |
| 42 | TCR9 alpha | SQELEQSPQSLIVQEGKNLTINCTSSKTLYGLYWYKQKYGEGLI |
| | chain mature | FLMMLQKGGEEKSHEKITAKLDEKKQQSSLHITASQPSHAGIYL |
| | | CGALASGGYQKVTFGIGTKLQVIPNIQNPDPAVYQLRDSKSSDK |
| | | SVCLFTDFDSQTNVSQSKDSDVYITDKTVLDMRSMDFKSNSAV |
| | | AWSNKSDFACANAFNNSIIPEDTFFPSPESSCDVKLVEKSFETDT |
| | | NLNFQNLSVIGFRILLLKVAGFNLLMTLRLWSS |
| 43 | TCR10 alpha | AQSVTQLGSHVSVSEGALVLLRCNYSSSVPPYLFWYVQYPNQG |
| | chain mature | LOLLLKYTSAATLVKGINGFEAEFKKSETSFHLTKPSAHMSDAA |
| | | EYFCAVSNMDSSYKLIFGSGTRLLVRPDIQNPDPAVYQLRDSKS |
| | | SDKSVCLFTDFDSQTNVSQSKDSDVYITDKTVLDMRSMDFKSN |
| | | SAVAWSNKSDFACANAFNNSIIPEDTFFPSPESSCDVKLVEKSFE |
| | | TDTNLNFQNLSVIGFRILLLKVAGFNLLMTLRLWSS |
| 44 | TCR11 alpha | QKEVEQNSGPLSVPEGAIASLNCTYSDRGSQSFFWYRQYSGKSP |
| | chain mature | ELIMFIYSNGDKEDGRFTAQLNKASQYVSLLIRDSQPSDSATYLC |
| | | AVPTGNTPLVFGKGTRLSVIANIQNPDPAVYQLRDSKSSDKSVC |
| | | LFTDFDSQTNVSQSKDSDVYITDKTVLDMRSMDFKSNSAVAWS |
| | | NKSDFACANAFNNSIIPEDTFFPSPESSCDVKLVEKSFETDTNLNF |
| | | QNLSVIGFRILLLKVAGFNLLMTLRLWSS |
| 45 | TCR1 beta chain | NAGVTQTPKFRVLKTGQSMTLLCAQDMNHEYMYWYRQDPGM |
| | mature | GLRLIHYSVGEGTTAKGEVPDGYNVSRLKKQNFLLGLESAAPS |
| | | QTSVYFCASSYPTGAYYGYTFGSGTRLTVVEDLNKVFPPEVAVF |
| | | EPSEAEISHTQKATLVCLATGFFPDHVELSWWVNGKEVHSGVS |
| | | TDPQPLKEQPALNDSRYCLSSRLRVSATFWQNPRNHFRCQVQF |
| | | YGLSENDEWTQDRAKPVTQIVSAEAWGRADCGFTSVSYQQGV |
| | | LSATILYEILLGKATLYAVLVSALVLMAMVKRKDF |
| 46 | TCR2 beta chain | DSGVTQTPKHLITATGQRVTLRCSPRSGDLSVYWYQQSLDQGL |
| | mature | QFLIQYYNGEERAKGNILERFSAQQFPDLHSELNLSSLELGDSAL |
| | | YFCASSAGDRGYEQYFGPGTRLTVTEDLKNVFPPEVAVFEPSEA |
| | | EISHTQKATLVCLATGFYPDHVELSWWVNGKEVHSGVSTDPQP |
| | | LKEQPALNDSRYCLSSRLRVSATFWQNPRNHFRCQVQFYGLSE |
| | | NDEWTQDRAKPVTQIVSAEAWGRADCGFTSESYQQGVLSATIL |
| | | YEILLGKATLYAVLVSALVLMAMVKRKDSRG |
| 47 | TCR3 beta chain | EADIYQTPRYLVIGTGKKITLECSQTMGHDKMYWYQQDPGME |
| | mature | LHLIHYSYGVNSTEKGDLSSESTVSRIRTEHFPLTLESARPSHTSQ |
| | | YLCASSGGYWDQENTEAFFGQGTRLTVVEDLNKVFPPEVAVFE |
| | | PSEAEISHTQKATLVCLATGFFPDHVELSWWVNGKEVHSGVST |
| | | DPQPLKEQPALNDSRYCLSSRLRVSATFWQNPRNHFRCQVQFY |
| | | GLSENDEWTQDRAKPVTQIVSAEAWGRADCGFTSVSYQQGVL |
| | | SATILYEILLGKATLYAVLVSALVLMAMVKRKDF |
| 48 | TCR4 beta chain | DAGVIQSPRHEVTEMGQEVTLRCKPISGHNSLFWYRQTMMRGL |
| | mature | ELLIYFNNNVPIDDSGMPEDRFSAKMPNASFSTLKIQPSEPRDSA |
| | | VYFCASSLGGQGGAGELFFGEGSRLTVLEDLKNVFPPEVAVFEP |
| | | SEAEISHTQKATLVCLATGFYPDHVELSWWVNGKEVHSGVSTD |
| | | PQPLKEQPALNDSRYCLSSRLRVSATFWQNPRNHFRCQVQFYG |
| | | LSENDEWTQDRAKPVTQIVSAEAWGRADCGFTSESYQQGVLSA |
| | | TILYEILLGKATLYAVLVSALVLMAMVKRKDSRG |
| 49 | TCR5 beta chain | EPEVTQTPSHQVTQMGQEVILRCVPISNHLYFYWYRQILGQKVE |
| | mature | FLVSFYNNEISEKSEIFDDQFSVERPDGSNFTLKIRSTKLEDSAMY |
| | | FCASSKVAGAIETQYFGPGTRLLVLEDLKNVFPPEVAVFEPSEAE |
| | | ISHTQKATLVCLATGFYPDHVELSWWVNGKEVHSGVSTDPQPL |
| | | KEQPALNDSRYCLSSRLRVSATFWQNPRNHFRCQVQFYGLSEN |
| | | DEWTQDRAKPVTQIVSAEAWGRADCGFTSESYQQGVLSATILY |
| | | EILLGKATLYAVLVSALVLMAMVKRKDSRG |
| 50 | TCR6 beta chain | DGGITQSPKYLFRKEGQNVTLSCEQNLNHDAMYWYRQDPGQG |
| | mature | LRLIYYSQIVNDFQKGDIAEGYSVSREKKESFPLTVTSAQKNPTA |
| | | FYLCASSKGIRYEQYFGPGTRLTVTEDLKNVFPPEVAVFEPSEAE |
| | | ISHTQKATLVCLATGFYPDHVELSWWVNGKEVHSGVSTDPQPL |
| | | KEQPALNDSRYCLSSRLRVSATFWQNPRNHFRCQVQFYGLSEN |
| | | DEWTQDRAKPVTQIVSAEAWGRADCGFTSESYQQGVLSATILY |
| | | EILLGKATLYAVLVSALVLMAMVKRKDSRG |
| 51 | TCR7 beta chain | EAQVTQNPRYLITVTGKKLTVTCSQNMNHEYMSWYRQDPGLG |
| | mature | LRQIYYSMNVEVTDKGDVPEGYKVSRKEKRNFPLILESPSPNQT |
| | | SLYFCASSARQGTNEKLFFGSGTQLSVLEDLNKVFPPEVAVFEPS |
| | | EAEISHTQKATLVCLATGFFPDHVELSWWVNGKEVHSGVSTDP |
| | | QPLKEQPALNDSRYCLSSRLRVSATFWQNPRNHFRCQVQFYGL |
| | | SENDEWTQDRAKPVTQIVSAEAWGRADCGFTSVSYQQGVLSA |
| | | TILYEILLGKATLYAVLVSALVLMAMVKRKDF |
| 52 | TCR8 beta chain | EAGVAQSPRYKIIEKRQSVAFWCNPISGHATLYWYQQILGQGPK |
| | mature | LLIQFQNNGVVDDSQLPKDRFSAERLKGVDSTLKIQPAKLEDSA |
| | | VYLCASSLVATWDEQFFGPGTRLTVLEDLKNVFPPEVAVFEPSE |
| | | AEISHTQKATLVCLATGFYPDHVELSWWVNGKEVHSGVSTDPQ |
| | | PLKEQPALNDSRYCLSSRLRVSATFWQNPRNHFRCQVQFYGLS |
| | | ENDEWTQDRAKPVTQIVSAEAWGRADCGFTSESYQQGVLSATI |
| | | LYEILLGKATLYAVLVSALVLMAMVKRKDSRG |
| 53 | TCR9 beta chain | NAGVTQTPKFRVLKTGQSMTLLCAQDMNHEYMYWYRQDPGM |
| | mature | GLRLIHYSVGEGTTAKGEVPDGYNVSRLKKQNFLLGLESAAPS |
| | | QTSVYFCASSPSGTPSGANVLTFGAGSRLTVLEDLKNVFPPEVA |
| | | VFEPSEAEISHTQKATLVCLATGFYPDHVELSWWVNGKEVHSG |
| | | VSTDPQPLKEQPALNDSRYCLSSRLRVSATFWQNPRNHFRCQV |
| | | QFYGLSENDEWTQDRAKPVTQIVSAEAWGRADCGFTSESYQQ |
| | | GVLSATILYEILLGKATLYAVLVSALVLMAMVKRKDSRG |
| 54 | TCR10 beta | GAVVSQHPSRVICKSGTSVKIECRSLDFQATTMFWYRQFPKKSL |
| | chain mature | MLMATSNEGSKATYEQGVEKDKFLINHASLTLSTLTVTSAHPE |
| | | DSSFYICSALPSGVDRLNTEAFFGQGTRLTVVEDLNKVFPPEVA |
| | | VFEPSEAEISHTQKATLVCLATGFFPDHVELSWWVNGKEVHSG |
| | | VSTDPQPLKEQPALNDSRYCLSSRLRVSATFWQNPRNHFRCQV |
| | | QFYGLSENDEWTQDRAKPVTQIVSAEAWGRADCGFTSVSYQQ |
| | | GVLSATILYEILLGKATLYAVLVSALVLMAMVKRKDF |
| 55 | TCR11 beta | DVKVTQSSRYLVKRTGEKVFLECVQDMDHENMFWYRQDPGL |
| | chain mature | GLRLIYFSYDVKMKEKGDIPEGYSVSREKKERFSLILESASTNQT |
| | | SMYLCAATGASRPTEAFFGQGTRLTVVEDLNKVFPPEVAVFEPS |
| | | EAEISHTQKATLVCLATGFFPDHVELSWWVNGKEVHSGVSTDP |
| | | QPLKEQPALNDSRYCLSSRLRVSATFWQNPRNHFRCQVQFYGL |
| | | SENDEWTQDRAKPVTQIVSAEAWGRADCGFTSVSYQQGVLSA |
| | | TILYEILLGKATLYAVLVSALVLMAMVKRKDF |
| 56 | TCR1 alpha | MLLLLVPVLEVIFTLGGTRAQSVTQLDSHVSVSEGTPVLLRCNY |
| | chain with signal | SSSYSPSLFWYVQHPNKGLQLLLKYTSAATLVKGINGFEAEFKK |
| | peptide | SETSFHLTKPSAHMSDAAEYFCVLKGGGNKLTFGTGTQLKVEL |
| | | NIQNPDPAVYQLRDSKSSDKSVCLFTDFDSQTNVSQSKDSDVYI |
| | | TDKTVLDMRSMDFKSNSAVAWSNKSDFACANAFNNSIIPEDTF |
| | | FPSPESSCDVKLVEKSFETDTNLNFQNLSVIGFRILLLKVAGFNL |
| | | LMTLRLWSS |
| 57 | TCR2 alpha | MEKMLECAFIVLWLQLGWLSGEDQVTQSPEALRLQEGESSSLN |
| | chain with signal | CSYTVSGLRGLFWYRQDPGKGPEFLFTLYSAGEEKEKERLKAT |
| | peptide | LTKKESFLHITAPKPEDSATYLCAVQAWGGGSQGNLIFGKGTKL |
| | | SVKPNIQNPDPAVYQLRDSKSSDKSVCLFTDFDSQTNVSQSKDS |
| | | DVYITDKTVLDMRSMDFKSNSAVAWSNKSDFACANAFNNSIIP |
| | | EDTFFPSPESSCDVKLVEKSFETDTNLNFQNLSVIGFRILLLKVA |
| | | GFNLLMTLRLWSS |
| 58 | TCR3 alpha | MISLRVLLVILWLQLSWVWSQRKEVEQDPGPFNVPEGATVAFN |
| | chain with signal | CTYSNSASQSFFWYRQDCRKEPKLLMSVYSSGNEDGRFTAQLN |
| | peptide | RASQYISLLIRDSKLSDSATYLCVVNGINRGSTLGRLYFGRGTQL |
| | | TVWPDIQNPDPAVYQLRDSKSSDKSVCLFTDFDSQTNVSQSKDS |
| | | DVYITDKTVLDMRSMDFKSNSAVAWSNKSDFACANAFNNSIIP |
| | | EDTFFPSPESSCDVKLVEKSFETDTNLNFQNLSVIGFRILLLKVA |
| | | GFNLLMTLRLWSS |
| 59 | TCR4 alpha | MKKHLTTFLVILWLYFYRGNGKNQVEQSPQSLIILEGKNCTLQC |
| | chain with signal | NYTVSPFSNLRWYKQDTGRGPVSLTIMTFSENTKSNGRYTATL |
| | peptide | DADTKQSSLHITASQLSDSASYICVVSAYARLMFGDGTQLVVKP |
| | | NIQNPDPAVYQLRDSKSSDKSVCLFTDFDSQTNVSQSKDSDVYI |
| | | TDKTVLDMRSMDFKSNSAVAWSNKSDFACANAFNNSIIPEDTF |
| | | FPSPESSCDVKLVEKSFETDTNLNFQNLSVIGFRILLLKVAGFNL |
| | | LMTLRLWSS |
| 60 | TCR5 alpha | METVLQVLLGILGFQAAWVSSQELEQSPQSLIVQEGKNLTINCT |
| | chain with signal | SSKTLYGLYWYKQKYGEGLIFLMMLQKGGEEKSHEKITAKLDE |
| | peptide | KKQQSSLHITASQPSHAGIYLCGADELGIQGAQKLVFGQGTRLTI |
| | | NPNIQNPDPAVYQLRDSKSSDKSVCLFTDFDSQTNVSQSKDSDV |
| | | YITDKTVLDMRSMDFKSNSAVAWSNKSDFACANAFNNSIIPEDT |
| | | FFPSPESSCDVKLVEKSFETDTNLNFQNLSVIGFRILLLKVAGFNL |
| | | LMTLRLWSS |
| 61 | TCR6 alpha | MISLRVLLVILWLQLSWVWSQRKEVEQDPGPFNVPEGATVAFN |
| | chain with signal | CTYSNSASQSFFWYRQDCRKEPKLLMSVYSSGNEDGRFTAQLN |
| | peptide | RASQYISLLIRDSKLSDSATYLCVVIEGNYGGATNKLIFGTGTLL |
| | | AVQPNIQNPDPAVYQLRDSKSSDKSVCLFTDFDSQTNVSQSKDS |
| | | DVYITDKTVLDMRSMDFKSNSAVAWSNKSDFACANAFNNSIIP |
| | | EDTFFPSPESSCDVKLVEKSFETDTNLNFQNLSVIGFRILLLKVA |
| | | GFNLLMTLRLWSS |
| 62 | TCR7 alpha | MSLSSLLKVVTASLWLGPGIAQKITQTQPGMFVQEKEAVTLDC |
| | chain with signal | TYDTSDQSYGLFWYKQPSSGEMIFLIYQGSYDEQNATEGRYSLN |
| | peptide | FQKARKSANLVISASQLGDSAMYFCAMREPYKYNNNDMRFGA |
| | | GTRLTVKPNIQNPDPAVYQLRDSKSSDKSVCLFTDFDSQTNVSQ |
| | | SKDSDVYITDKTVLDMRSMDFKSNSAVAWSNKSDFACANAFN |
| | | NSIIPEDTFFPSPESSCDVKLVEKSFETDTNLNFQNLSVIGFRILLL |
| | | KVAGFNLLMTLRLWSS |
| 63 | TCR8 alpha | MACPGFLWALVISTCLEFSMAQTVTQSQPEMSVQEAETVTLSC |
| | chain with signal | TYDTSESDYYLFWYKQPPSRQMILVIRQEAYKQQNATENRFSV |
| | peptide | NFQKAAKSFSLKISDSQLGDAAMYFCAYPLVNQGGKLIFGQGT |
| | | ELSVKPNIQNPDPAVYQLRDSKSSDKSVCLFTDFDSQTNVSQSK |
| | | DSDVYITDKTVLDMRSMDFKSNSAVAWSNKSDFACANAFNNSI |
| | | IPEDTFFPSPESSCDVKLVEKSFETDTNLNFQNLSVIGFRILLLKV |
| | | AGFNLLMTLRLWSS |
| 64 | TCR9 alpha | METVLQVLLGILGFQAAWVSSQELEQSPQSLIVQEGKNLTINCT |
| | chain with signal | SSKTLYGLYWYKQKYGEGLIFLMMLQKGGEEKSHEKITAKLDE |
| | peptide | KKQQSSLHITASQPSHAGIYLCGALASGGYQKVTFGIGTKLQVIP |
| | | NIQNPDPAVYQLRDSKSSDKSVCLFTDFDSQTNVSQSKDSDVYI |
| | | TDKTVLDMRSMDFKSNSAVAWSNKSDFACANAFNNSIIPEDTF |
| | | FPSPESSCDVKLVEKSFETDTNLNFQNLSVIGFRILLLKVAGFNL |
| | | LMTLRLWSS |
| 65 | TCR10 alpha | MLLLLVPVLEVIFTLGGTRAQSVTQLGSHVSVSEGALVLLRCNY |
| | chain with signal | SSSVPPYLFWYVQYPNQGLQLLLKYTSAATLVKGINGFEAEFKK |
| | peptide | SETSFHLTKPSAHMSDAAEYFCAVSNMDSSYKLIFGSGTRLLVR |
| | | PDIQNPDPAVYQLRDSKSSDKSVCLFTDFDSQTNVSQSKDSDVY |
| | | ITDKTVLDMRSMDFKSNSAVAWSNKSDFACANAFNNSIIPEDTF |
| | | FPSPESSCDVKLVEKSFETDTNLNFQNLSVIGFRILLLKVAGFNL |
| | | LMTLRLWSS |
| 66 | TCR11 alpha | MKSLRVLLVILWLQLSWVWSQQKEVEQNSGPLSVPEGAIASLN |
| | chain with signal | CTYSDRGSQSFFWYRQYSGKSPELIMFIYSNGDKEDGRFTAQLN |
| | peptide | KASQYVSLLIRDSQPSDSATYLCAVPTGNTPLVFGKGTRLSVIA |
| | | NIQNPDPAVYQLRDSKSSDKSVCLFTDFDSQTNVSQSKDSDVYI |
| | | TDKTVLDMRSMDFKSNSAVAWSNKSDFACANAFNNSIIPEDTF |
| | | FPSPESSCDVKLVEKSFETDTNLNFQNLSVIGFRILLLKVAGFNL |
| | | LMTLRLWSS |
| 67 | TCR1 beta chain | MSLGLLCCGAFSLLWAGPVNAGVTQTPKFRVLKTGQSMTLLC |
| | with signal | AQDMNHEYMYWYRQDPGMGLRLIHYSVGEGTTAKGEVPDGY |
| | peptide | NVSRLKKQNFLLGLESAAPSQTSVYFCASSYPTGAYYGYTFGSG |
| | | TRLTVVEDLNKVFPPEVAVFEPSEAEISHTQKATLVCLATGFFPD |
| | | HVELSWWVNGKEVHSGVSTDPQPLKEQPALNDSRYCLSSRLRV |
| | | SATFWQNPRNHFRCQVQFYGLSENDEWTQDRAKPVTQIVSAEA |
| | | WGRADCGFTSVSYQQGVLSATILYEILLGKATLYAVLVSALVL |
| | | MAMVKRKDF |
| 68 | TCR2 beta chain | MGFRLLCCVAFCLLGAGPVDSGVTQTPKHLITATGQRVTLRCSP |
| | with signal | RSGDLSVYWYQQSLDQGLQFLIQYYNGEERAKGNILERFSAQQ |
| | peptide | FPDLHSELNLSSLELGDSALYFCASSAGDRGYEQYFGPGTRLTV |
| | | TEDLKNVFPPEVAVFEPSEAEISHTQKATLVCLATGFYPDHVELS |
| | | WWVNGKEVHSGVSTDPQPLKEQPALNDSRYCLSSRLRVSATF |
| | | WQNPRNHFRCQVQFYGLSENDEWTQDRAKPVTQIVSAEAWGR |
| | | ADCGFTSESYQQGVLSATILYEILLGKATLYAVLVSALVLMAM |
| | | VKRKDSRG |
| 69 | TCR3 beta chain | MTIRLLCYVGFYFLGAGLMEADIYQTPRYLVIGTGKKITLECSQ |
| | with signal | TMGHDKMYWYQQDPGMELHLIHYSYGVNSTEKGDLSSESTVS |
| | peptide | RIRTEHFPLTLESARPSHTSQYLCASSGGYWDQENTEAFFGQGT |
| | | RLTVVEDLNKVFPPEVAVFEPSEAEISHTQKATLVCLATGFFPD |
| | | HVELSWWVNGKEVHSGVSTDPQPLKEQPALNDSRYCLSSRLRV |
| | | SATFWQNPRNHFRCQVQFYGLSENDEWTQDRAKPVTQIVSAEA |
| | | WGRADCGFTSVSYQQGVLSATILYEILLGKATLYAVLVSALVL |
| | | MAMVKRKDF |
| 70 | TCR4 beta chain | MDSWTFCCVSLCILVAKHTDAGVIQSPRHEVTEMGQEVTLRCK |
| | with signal | PISGHNSLFWYRQTMMRGLELLIYFNNNVPIDDSGMPEDRFSAK |
| | peptide | MPNASFSTLKIQPSEPRDSAVYFCASSLGGQGGAGELFFGEGSR |
| | | LTVLEDLKNVFPPEVAVFEPSEAEISHTQKATLVCLATGFYPDH |
| | | VELSWWVNGKEVHSGVSTDPQPLKEQPALNDSRYCLSSRLRVS |
| | | ATFWQNPRNHFRCQVQFYGLSENDEWTQDRAKPVTQIVSAEA |
| | | WGRADCGFTSESYQQGVLSATILYEILLGKATLYAVLVSALVL |
| | | MAMVKRKDSRG |
| 71 | TCR5 beta chain | MDTWLVCWAIFSLLKAGLTEPEVTQTPSHQVTQMGQEVILRCV |
| | with signal | PISNHLYFYWYRQILGQKVEFLVSFYNNEISEKSEIFDDQFSVER |
| | peptide | PDGSNFTLKIRSTKLEDSAMYFCASSKVAGAIETQYFGPGTRLL |
| | | VLEDLKNVFPPEVAVFEPSEAEISHTQKATLVCLATGFYPDHVE |
| | | LSWWVNGKEVHSGVSTDPQPLKEQPALNDSRYCLSSRLRVSAT |
| | | FWQNPRNHFRCQVQFYGLSENDEWTQDRAKPVTQIVSAEAWG |
| | | RADCGFTSESYQQGVLSATILYEILLGKATLYAVLVSALVLMA |
| | | MVKRKDSRG |
| 72 | TCR6 beta chain | MSNQVLCCVVLCFLGANTVDGGITQSPKYLFRKEGQNVTLSCE |
| | with signal | QNLNHDAMYWYRQDPGQGLRLIYYSQIVNDFQKGDIAEGYSV |
| | peptide | SREKKESFPLTVTSAQKNPTAFYLCASSKGIRYEQYFGPGTRLTV |
| | | TEDLKNVFPPEVAVFEPSEAEISHTQKATLVCLATGFYPDHVELS |
| | | WWVNGKEVHSGVSTDPQPLKEQPALNDSRYCLSSRLRVSATF |
| | | WQNPRNHFRCQVQFYGLSENDEWTQDRAKPVTQIVSAEAWGR |
| | | ADCGFTSESYQQGVLSATILYEILLGKATLYAVLVSALVLMAM |
| | | VKRKDSRG |
| 73 | TCR7 beta chain | MGPQLLGYVVLCLLGAGPLEAQVTQNPRYLITVTGKKLTVTCS |
| | with signal | QNMNHEYMSWYRQDPGLGLRQIYYSMNVEVTDKGDVPEGYK |
| | peptide | VSRKEKRNFPLILESPSPNQTSLYFCASSARQGTNEKLFFGSGTQ |
| | | LSVLEDLNKVFPPEVAVFEPSEAEISHTQKATLVCLATGFFPDHV |
| | | ELSWWVNGKEVHSGVSTDPQPLKEQPALNDSRYCLSSRLRVSA |
| | | TFWQNPRNHFRCQVQFYGLSENDEWTQDRAKPVTQIVSAEAW |
| | | GRADCGFTSVSYQQGVLSATILYEILLGKATLYAVLVSALVLM |
| | | AMVKRKDF |
| 74 | TCR8 beta chain | MGTRLLCWAALCLLGAELTEAGVAQSPRYKIIEKRQSVAFWCN |
| | with signal | PISGHATLYWYQQILGQGPKLLIQFQNNGVVDDSQLPKDRFSAE |
| | peptide | RLKGVDSTLKIQPAKLEDSAVYLCASSLVATWDEQFFGPGTRLT |
| | | VLEDLKNVFPPEVAVFEPSEAEISHTQKATLVCLATGFYPDHVE |
| | | LSWWVNGKEVHSGVSTDPQPLKEQPALNDSRYCLSSRLRVSAT |
| | | FWQNPRNHFRCQVQFYGLSENDEWTQDRAKPVTQIVSAEAWG |
| | | RADCGFTSESYQQGVLSATILYEILLGKATLYAVLVSALVLMA |
| | | MVKRKDSRG |
| 75 | TCR9 beta chain | MSLGLLCCGAFSLLWAGPVNAGVTQTPKFRVLKTGQSMTLLC |
| | with signal | AQDMNHEYMYWYRQDPGMGLRLIHYSVGEGTTAKGEVPDGY |
| | peptide | NVSRLKKQNFLLGLESAAPSQTSVYFCASSPSGTPSGANVLTFG |
| | | AGSRLTVLEDLKNVFPPEVAVFEPSEAEISHTQKATLVCLATGF |
| | | YPDHVELSWWVNGKEVHSGVSTDPQPLKEQPALNDSRYCLSSR |
| | | LRVSATFWQNPRNHFRCQVQFYGLSENDEWTQDRAKPVTQIVS |
| | | AEAWGRADCGFTSESYQQGVLSATILYEILLGKATLYAVLVSAL |
| | | VLMAMVKRKDSRG |
| 76 | TCR10 beta | MLLLLLLLGPGSGLGAVVSQHPSRVICKSGTSVKIECRSLDFQAT |
| | chain with signal | TMFWYRQFPKKSLMLMATSNEGSKATYEQGVEKDKFLINHAS |
| | peptide | LTLSTLTVTSAHPEDSSFYICSALPSGVDRLNTEAFFGQGTRLTV |
| | | VEDLNKVFPPEVAVFEPSEAEISHTQKATLVCLATGFFPDHVELS |
| | | WWVNGKEVHSGVSTDPQPLKEQPALNDSRYCLSSRLRVSATF |
| | | WQNPRNHFRCQVQFYGLSENDEWTQDRAKPVTQIVSAEAWGR |
| | | ADCGFTSVSYQQGVLSATILYEILLGKATLYAVLVSALVLMAM |
| | | VKRKDF |
| 77 | TCR11 beta | MGIRLLCRVAFCFLAVGLVDVKVTQSSRYLVKRTGEKVFLECV |
| | chain with signal | QDMDHENMFWYRQDPGLGLRLIYFSYDVKMKEKGDIPEGYSV |
| | peptide | SREKKERFSLILESASTNQTSMYLCAATGASRPTEAFFGQGTRLT |
| | | VVEDLNKVFPPEVAVFEPSEAEISHTQKATLVCLATGFFPDHVE |
| | | LSWWVNGKEVHSGVSTDPQPLKEQPALNDSRYCLSSRLRVSAT |
| | | FWQNPRNHFRCQVQFYGLSENDEWTQDRAKPVTQIVSAEAWG |
| | | RADCGFTSVSYQQGVLSATILYEILLGKATLYAVLVSALVLMA |
| | | MVKRKDF |

In preferred embodiments, the TCR comprises an alpha chain having a CDR3 set forth in SEQ ID Nos: 12-22 and a beta chain having a CDR3 set forth in SEQ ID Nos:23-33. The CDR3 region may be determined by commercially available software (e.g. Cellranger; 10× Genomics, Pleasanton, CA). The TCR alpha chain may comprise a sequence at least at least 95%, at least 96%, at least 97%, at least 98%, at least 99%, or 100% identical to the sequence set forth in any of SEQ ID Nos:34-44. The TCR beta chain may comprise a sequence at least 95%, at least 96%, at least 97%, at least 98%, at least 99%, or 100% identical to the sequence set forth in any of SEQ ID Nos:45-55. Methods of determining the identity between two sequences are well-known in the art, e.g., BLAST or Geneious. In certain embodiments, the C-terminal or N-terminal 1, 2, 3, 4, 5, 6, 7, 8, 9, or 10 residues of any of the sequences set forth is any of SEQ ID Nos:35-45 or any of the sequences set forth in any of SEQ ID Nos:45-55 may be truncated or removed. Exemplary TCRs and the corresponding alpha and beta chain CDR3 and full-length SEQ ID Nos. are provided in Table 2.

TABLE 2
Alpha and beta chain CDR3 and alpha and beta
chain mature full length sequence IDs.

| | | | Alpha mature | Beta mature |
|---|---|---|---|---|
| TCR | Alpha CDR3 | Beta CDR3 | full-length | full-length |
| 1 | SEQ ID NO: 12 | SEQ ID NO: 23 | SEQ ID NO: 34 | SEQ ID NO: 45 |
| 2 | SEQ ID NO: 13 | SEQ ID NO: 24 | SEQ ID NO: 35 | SEQ ID NO: 46 |
| 3 | SEQ ID NO: 14 | SEQ ID NO: 25 | SEQ ID NO: 36 | SEQ ID NO: 47 |
| 4 | SEQ ID NO: 15 | SEQ ID NO: 26 | SEQ ID NO: 37 | SEQ ID NO: 48 |
| 5 | SEQ ID NO: 16 | SEQ ID NO: 27 | SEQ ID NO: 38 | SEQ ID NO: 49 |
| 6 | SEQ ID NO: 17 | SEQ ID NO: 28 | SEQ ID NO: 39 | SEQ ID NO: 50 |
| 7 | SEQ ID NO: 18 | SEQ ID NO: 29 | SEQ ID NO: 40 | SEQ ID NO: 51 |
| 8 | SEQ ID NO: 19 | SEQ ID NO: 30 | SEQ ID NO: 41 | SEQ ID NO: 52 |
| 9 | SEQ ID NO: 20 | SEQ ID NO: 31 | SEQ ID NO: 42 | SEQ ID NO: 53 |
| 10 | SEQ ID NO: 21 | SEQ ID NO: 32 | SEQ ID NO: 43 | SEQ ID NO: 54 |
| 11 | SEQ ID NO: 22 | SEQ ID NO: 33 | SEQ ID NO: 44 | SEQ ID NO: 55 |

In certain embodiments, the variable domain of a TCR alpha or beta chain may be fused to a non-TCR polypeptide. The exemplary alpha and beta chain variable domains may be used to create a soluble TCR capable of binding the DCAF4L2-derived peptide in the context of an HLA molecule. The soluble TCRs may be in single chain format wherein the alpha and beta variable domains are connected by a linker. A disulfide bond may be introduced between the alpha and beta chains to increase stability. The soluble TCRs may be fused or connected to therapeutic or imaging agent. Exemplary TCRs and the corresponding alpha and beta variable regions are provided in Table 3.

TABLE 3
alpha and beta variable regions sequence IDs.

| TCR | Alpha variable domain | Beta variable domain |
|---|---|---|
| 1 | Amino acids 1-113 SEQ ID NO: 34 | Amino acids 1-114 SEQ ID NO: 45 |
| 2 | Amino acids 1-117 SEQ ID NO: 35 | Amino acids 1-114 SEQ ID NO: 46 |
| 3 | Amino acids 1-118 SEQ ID NO: 36 | Amino acids 1-113 SEQ ID NO: 47 |
| 4 | Amino acids 1-112 SEQ ID NO: 37 | Amino acids 1-113 SEQ ID NO: 48 |
| 5 | Amino acids 1-107 SEQ ID NO: 38 | Amino acids 1-111 SEQ ID NO: 49 |
| 6 | Amino acids 1-113 SEQ ID NO: 39 | Amino acids 1-114 SEQ ID NO: 50 |
| 7 | Amino acids 1-112 SEQ ID NO: 40 | Amino acids 1-116 SEQ ID NO: 51 |
| 8 | Amino acids 1-116 SEQ ID NO: 41 | Amino acids 1-113 SEQ ID NO: 52 |
| 9 | Amino acids 1-109 SEQ ID NO: 42 | Amino acids 1-111 SEQ ID NO: 53 |
| 10 | Amino acids 1-112 SEQ ID NO: 43 | Amino acids 1-114 SEQ ID NO: 54 |
| 11 | Amino acids 1-113 SEQ ID NO: 44 | Amino acids 1-112 SEQ ID NO: 55 |

The TCR alpha or beta variable domain may comprise a sequence at least 95%, at least 96%, at least 97%, at least 98%, at least 99%, or 100% identical to any of the sequences specified in Table 2. The TCR beta chain may comprise a sequence at least 80%, at least 85%, at least 90%, at least 95%, at least 96%, at least 97%, at least 98%, at least 99%, or 100% identical to the sequence set forth is any of SEQ ID Nos:45-55. In certain embodiments, the C-terminal or N-terminal 1, 2, 3, 4, 5, 6, 7, 8, 9, or 10 residues of any of the sequences specified in Table 2 may be truncated or removed.

Although recognition of the target peptide in the context of HLA is required for efficacy, for safety purposes, in some embodiments it is preferred that the TCR lacks cross-reactivity with structurally similar peptides when presented by HLA-A*02:01 or with HLA molecules of other allotypes. The cross-reactivity and alloreactivity of the exemplary TCRs described herein are provided in the Examples. Thus, the exemplary TCRs not only are able to recognize the DCAF4L2 peptide in the context of HLA-A*02:01 as expressed on tumor cells and activate a T cell recombinantly expressing the TCR against the tumor cell but also fail to activate or have minimal activation when the recombinant T cell is presented with peptides in the context of HLA-A*02:01 or other HLA molecules that are expressed on normal tissue.

Further embodiments of the present invention include nucleic acids encoding a TCR alpha variable domain, a TCR beta variable domain, or a TCR alpha variable domain and a TCR beta variable domain described herein. In particular embodiments, the nucleic acid encodes one or more of the alpha or beta variable domains set forth in Table 2. In certain embodiments, the nucleic acid encodes both alpha and beta variable domains of TCR1, TCR2, TCR3, TCR4, TCR5, TCR6, TCR7, TCR8, TCR9, TCR10, or TCR11. In preferred embodiments, the nucleic acid encoding the TCR alpha chain variable domain, TCR beta chain variable domain, or TCR alpha chain variable domain and beta chain variable domain is an expression vector wherein the TCR alpha chain variable domain, TCR beta chain variable domain, or TCR alpha chain variable domain and beta chain variable domain is operably linked to a promoter.

The TCR alpha variable domain and beta variable domain may be co-transcribed from the same promoter. For embodiments wherein the alpha variable domain and beta variable domain are linked within a fusion protein, the domains may be co-translated within a single polypeptide as well. In embodiments wherein the alpha domain and beta domain are within separate polypeptides, it is useful to include an internal ribosome entry site (IRES) between the alpha variable domain and beta variable domain coding regions within the expression vector.

Also provided herein are nucleic acids encoding a TCR alpha chain, a TCR beta chain, or a TCR alpha and TCR beta chain described herein. In particular embodiments, the nucleic acid encodes one or more the alpha or beta chains set forth in Table 1. The encoded alpha or beta chain may be full-length or mature. When mature, i.e., lacking the nature leader sequence associated with that alpha or beta chain, it is preferred that a nucleic acid encoding a signal or leader sequence is operably connected to the nucleic acid encoding the alpha chain or beta chain such that, when translated, the leader sequence directs the alpha or beta chain to the endoplasmic reticulum.

In certain embodiments, the nucleic acid encodes both alpha and beta chains of TCR1, TCR2, TCR3, TCR4, TCR5, TCR6, TCR7, TCR8, TCR9, TCR10, or TCR11. In preferred embodiments, the nucleic acid encoding the TCR alpha chain, TCR beta chain, or TCR alpha chain and beta chain is an expression vector wherein the TCR alpha chain, TCR beta chain, or TCR alpha chain and beta chain is operably linked to a promoter.

The TCR alpha chain and beta chain may be co-transcribed from the same promoter. In such embodiments, it is useful to include an internal ribosome entry site (IRES) between the alpha chain and beta chain coding regions within the expression vector.

The expression vectors of the present invention include, but are not limited to, retroviral or lentiviral vectors. The expression vector may further encode one or more additional proteins besides the TCR alpha chain and/or beta chain. In certain embodiments, the expression vector encodes one or more cytokines. In preferred embodiments, the cytokine is a T cell growth factor such as IL-2, IL-7, IL-12, IL-15, IL-18, or IL-21, along with combinations thereof. Because cytokines can have systemic effects, when the expression vector encoding the cytokine is used to produce a cell for adoptive cell therapy, it is preferred that the cytokine expression is controlled by an inducible promoter. In certain embodiments, the promoter is a composite promoter containing six NFAT (nuclear factor of activated T cells) response elements linked to a minimal IL-2 promoter and the cytokine is IL-12 or a variant thereof. Use of a composite promoter containing six NFAT (nuclear factor of activated T cells) response elements linked to a minimal IL-2 promoter to express IL-12 is described in U.S. Pat. No. 8,556,882.

Provided herein are cells recombinantly expressing an exemplary TCR described herein. Said recombinant cells may comprise one or more expression vectors encoding and expressing a TCR alpha chain, a TCR beta chain, a TCR alpha and beta chain, a TCR alpha variable domain, a TCR beta variable domain, or TCR alpha and beta variable domains. In preferred embodiments, the cell recombinantly expresses TCR1, TCR2, TCR3, TCR4, TCR5, TCR6, TCR7, TCR8, TCR9, TCR10, or TCR11. In certain embodiments, the cell further expresses one or more recombinant cytokines. In preferred embodiments, the cytokine is IL-12 or a variant thereof and said expression is controlled by an inducible promoter, e.g., an NFAT driven promoter.

In certain embodiments, the cells are derived from a sample taken from a cancer patient. Cells, such as T cells or NKT cells, are isolated from the sample and expanded. In certain embodiments, progenitor cells are isolated and matured to the desired cell type. The cells are transfected/transformed with one or more vectors, e.g., lentiviral vectors, encoding the components of the TCR along with any additional polypeptides, e.g., IL-12 or a variant thereof. Such cells may be used for adoptive cell therapy for the cancer patient from whom they were derived.

In other embodiments, a cell line recombinantly expresses a soluble TCR. The soluble TCR may be a fusion protein with an anti-CD3 antigen binding protein such as an scFv.

Provided herein are methods of treating a disease or disorder wherein cells associated with the disease or disorder express DCAF4L2. In preferred embodiments, the cells present the DCAF4L2 derived peptide ILQDGQFLV (SEQ ID NO:1) in the context of an HLA class I molecule, preferably HLA-A2, particularly HLA-A*02:01. Exemplary diseases or disorders that may be treated with the soluble TCRs or recombinant cells of the present invention include hematological or solid tumors. Preferred diseases and disorders include hepatocellular carcinoma (HCC).

For certain treatments, a biopsy of the tumor is tested for expression of DCAF4L2. The tumor may also be tested for expression of an appropriate HLA molecule that is recognized by a TCR of the present invention when presenting the DCAF4L2-derived peptide. Patients whose tumor express DCAF4L2 and are of the appropriate HLA haplotype may be administered a soluble TCR or recombinant cell of the present invention.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more; the terms “a” (or “an”), “one or more,” and “at least one” can be used interchangeably herein. The term “or” should be understood to encompass items in the alternative or together unless context unambiguously requires otherwise. The term “and/or” should be understood to encompass each item in a list (individually), any combination of items a list, and all items in a list together. As used herein, “can be” or “can” indicates something envisaged by the inventors that is functional and available as part of the subject matter provided.

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. All documents, or portions of documents, cited in this application, including but not limited to patents, patent applications, articles, books, and treatises, are hereby expressly incorporated by reference.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

The following examples, both actual and prophetic, are provided for the purpose of illustrating specific embodiments or features of the present invention and are not intended to limit its scope.

### Example 1—Dcaf4L2 is Overexpressed in Hepatocellular Carcinoma with Highly Restricted Normal Tissue Expression

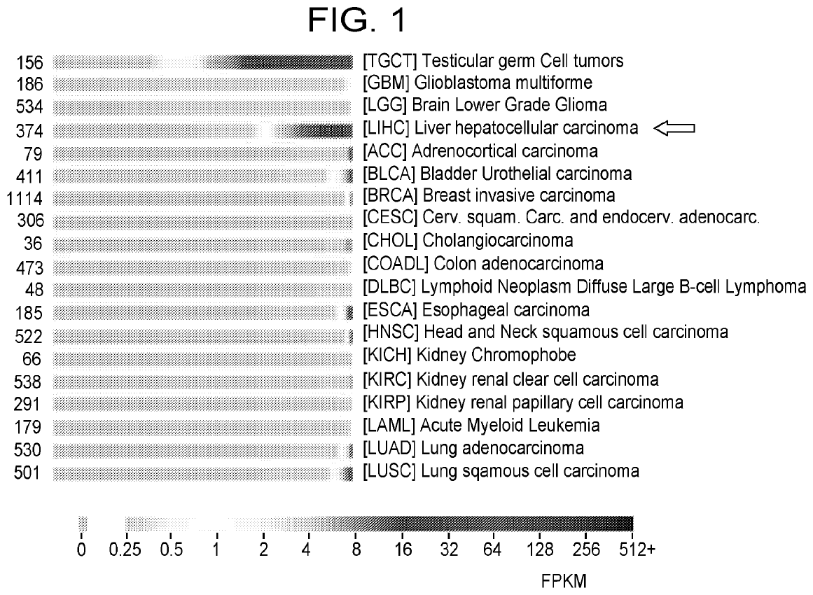

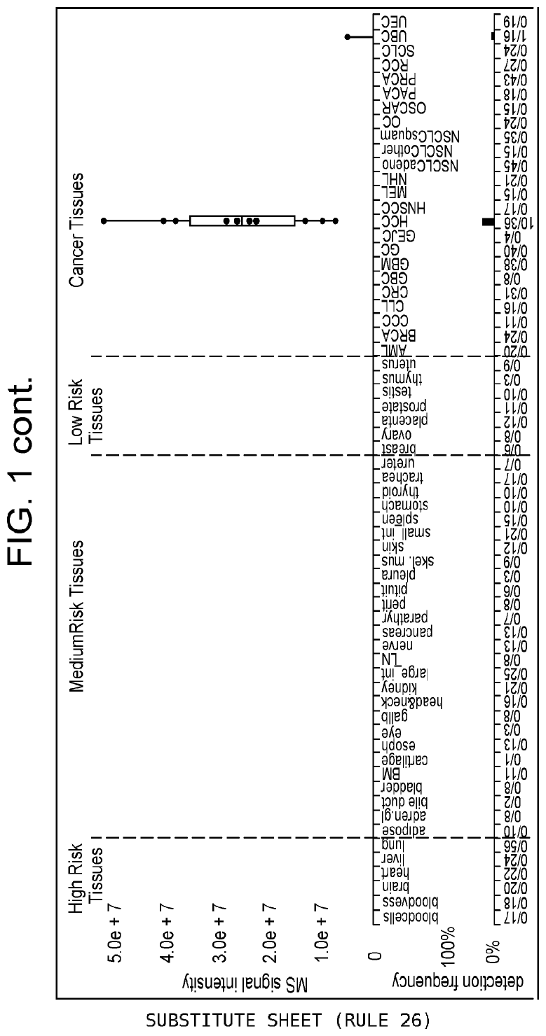

The Cancer Genome Atlas Program (TCGA) data demonstrate that DCAF4L2 is highly expressed in a subset of hepatocellular carcinomas, with approximately 29% percent of samples expressing DCAF4L2 at an FPKM level≥1 (FIG. 1A). Importantly, as pMHC targets, DCAF4L2 peptide (ILQDGQFLV) presentation on HLA-A*02:01 was validated by mass spectrometry (MS). The MS data using various tumors and normal tissues (Immatics, Houston, TX) demonstrated that DCAF4L2 peptide-MHC (ILQDGQFLV-HLA-A*02:01) expression is very specific for hepatocellular carcinoma (HCC) and not detected in normal healthy tissues (FIG. 1B). DCAF4L2 pMHC complex was observed by MS in 10/36 (˜28%) of HCC samples by MS. The DCAF4L2 peptide ILQDGQFLV (SEQ ID NO:1) corresponds to amino acid residues 278-286 of the DCAF4L2 protein.

### Example 2—Identification of Dcaf4L2 Pmhc-Specific Tcrs

The process to identify and select lead clinical TCR candidates is outlined below. First, using a TCR discovery platform based on ex vivo stimulation and scRNAseq, 32 dominant DCAF4L2 pMHC-specific TCRs were identified from naturally occurring T-cell clones isolated from 38 healthy HLA-A*02:01+ donors. Using Jurkat activation assays, 11 TCR candidates were selected for further validation. Based on these 11 TCR sequences, 11 TCR-T cells per donor were generated by transduction of primary pan-T cells isolated from 3 donors with lentivirus carrying individual TCRs. Those TCR-T cells were further evaluated by various functional assays including potency (cytotoxicity) tests against the T2 cell line that was pulsed with target peptides. To further enhance the in vivo efficacy and decrease clinical doses, the top TCRs were manufactured in a TCR-T-IL12 lentiviral construct, where the IL12 payload expression is induced upon TCR activation under a NFAT response-driven promoter. Therefore, only when TCR-T cells bind to their pMHC targets (DCAF4L2-HLA-A*02:01) in tumors, the IL12 can be produced. The TCR-T-IL12 cells generated were further evaluated by various functional assays, including potency tests with multiple cancer cell lines, cross-reactivity screens with a full panel of similar peptides, normal cell cytotoxicity screens, and alloreactivity screens. Based on the data from these evaluations, one lead clinical TCR candidate was selected.

DCAF4L2 pMHC-Specific TCRs can be Identified from Rare T Cell Clones Isolated from Healthy Donor PBMCs

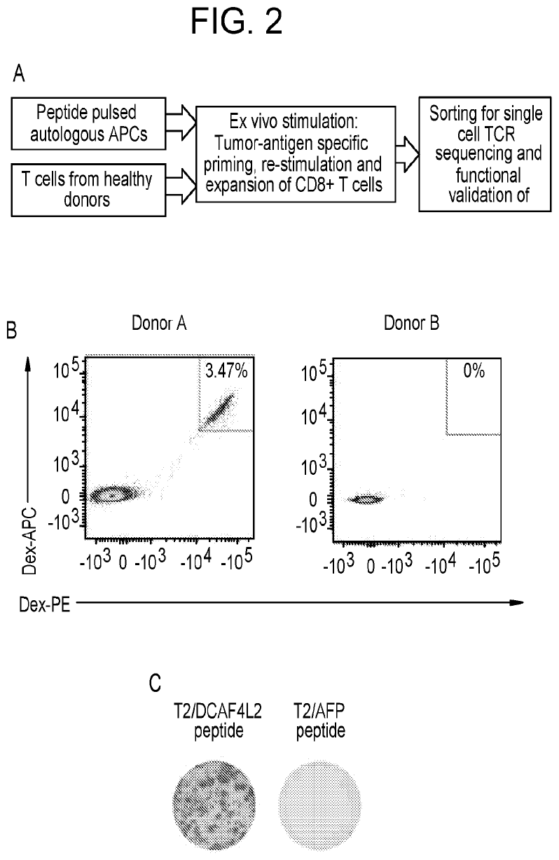

Difficulties in identifying tumor antigen-specific TCRs have hampered the development of TCR-mediated immunotherapies. Despite these challenges, we have successfully developed a TCR discovery platform by which the tumor antigen pMHC-specific TCRs can be identified from rare T cell clones isolated from healthy donor PBMCs. The frequencies of DCAF4L2 pMHC-reactive T cells in PBMCs from healthy HLA-A*02:01+ donors were extremely low, which were typically ˜0% dextramer+ T cells. Dextramer (Dex) is a multimer of peptide-MHC complexes that can specifically bind to TCRs, and therefore can be used to isolate antigen (pMHC)-specific T cells. First, in order to expand the rare tumor antigen-specific T clones, we used healthy HLA-A*02:01+ donor PBMCs to isolate T cells and autologous antigen-presenting cells (APCs) such as monocyte-derived dendritic cells and activated B cells (FIG. 2A). Upon co-culture of T cells with the autologous APCs pulsed with target peptides, these T cells went through multiple steps of ex vivo stimulations where tumor antigen pMHC-specific priming, restimulation, and expansion of pMHC-specific T cells occur (FIG. 2A). After multiple antigen restimulations, a population of DCAF4L2 pMHC dextramer+(Dex+) T cells (DCAF4L2 pMHC-reactive T cells) were detected. After 2-4 rounds of antigen restimulations, the DCAF4L2 pMHC-specific T cell population was further enriched and validated by both dextramer-PE and dextramer-APC stains (FIG. 2B). The Dex+CD8+ T cells were then sorted for single cell RNAseq to identify the sequences of TCRα and TCRβ chains. Additionally, the sorted Dex+CD8+ T cells were validated for DCAF4L2 antigen-specific activation by an IFNγ ELISPOT assay using peptide-loaded T2 cells (FIG. 2C). This TCR discovery platform led to the identification of 32 dominant DCAF4L2 pMHC-specific TCRs from 38 healthy HLA-A*02:01+ donors. Importantly, the TCRs identified from healthy donor blood have been through thymic natural selection in the human body (in the medulla of the thymus) to eliminate self-reactive TCRs, unlike affinity-enhanced TCRs or bispecific antibodies. Therefore, it is contemplated that the risk of off-targets for our TCRs is fairly low, which was confirmed by our safety assessment assays (described below).

Selection of Top DCAF4L2 pMHC-Specific TCR-T Cells

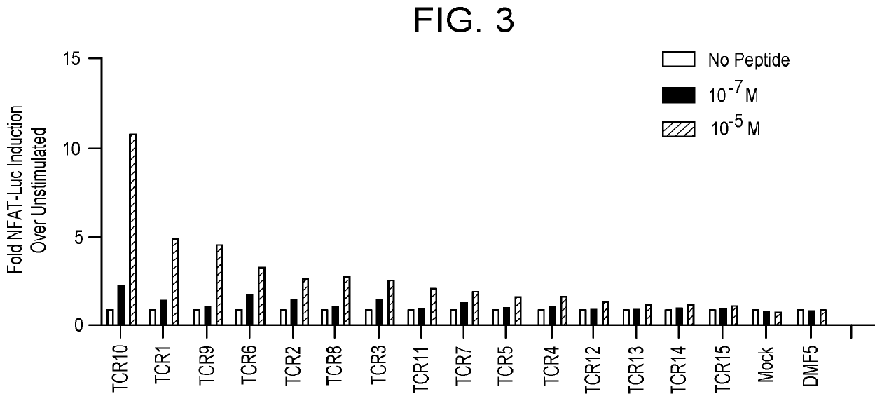

Out of 32 dominant DCAF4L2 pMHC-specific TCRs identified from a screen of 32 healthy HLA-A*02:01+ donors, 11 TCR candidates were selected by a Jurkat activation assay. Lentivirus carrying individual TCRs and GFP were transduced into a Jurkat TCR KO reporter cell line expressing CD8a constitutively and Renilla luciferase that is regulated by TCR activation under an NFAT response element driven promoter. The activity of individual TCRs was measured as the fold change of the luciferase activity in the presence of T2 cells loaded with the DCAF4L2 peptide compared to T2 cells with vehicle only (FIG. 3). From these data, 11 top TCR candidates were identified.

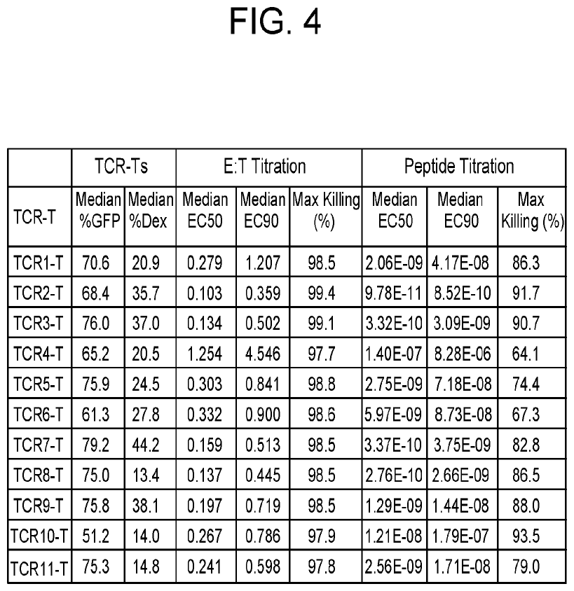

Human primary pan T cells isolated from 3 donors were transduced with lentivirus expressing an individual TCR out of 11 candidate DCAF4L2-specific TCRs (FIG. 4). GFP and DCAF4L2 dextramer frequencies were detected by flow cytometry. The similar median GFP frequencies among the TCR-Ts indicate a similar degree of successful transduction, though dextramer staining indicates variable TCR surface expression. Potencies of the 11 candidate DCAF4L2 TCR-Ts were assessed in T cell dependent cellular cytotoxicity (TDCC) assays with E:T (effector: target cell ratio) titration and DCAF4L2 peptide titration. Luciferase-expressing T2 (T2.luc) cells endogenously express HLA-A*02:01 and were pulsed with DCAF4L2 peptide to serve as target cells in the co-culture assay (FIG. 4). Based on TCR expression and potency data, TCR1, TCR2, TCR3, TCR8, and TCR9 were selected for further validation.

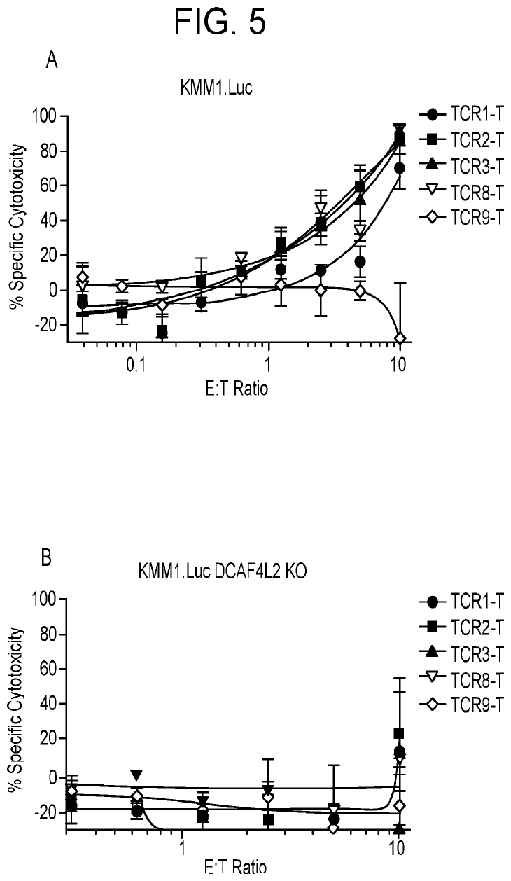

These top five TCR-Ts were subsequently evaluated in a TDCC assay using DCAF4L2+ HLA-A*02:01+ cancer cell lines (e.g., KMM1.Luc), allowing for evaluation of cytotoxic activity of the TCR-Ts against target cancer cells (FIG. 5A). TCR2 and TCR3 demonstrated potent killing against the KMM1.Luc cancer cell line. Importantly, cytotoxicity of these TCR-Ts was abrogated upon knockout of DCAF4L2 in the cancer cell line (KMM1.Luc DCAF4L2 KO), confirming that the cytolytic activity of TCR-T cells is dependent on DCAF4L2 target expression (FIG. 5B).

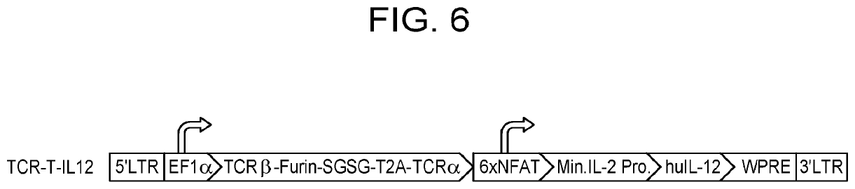

Based on high potency and transduced expression levels of TCR2 and TCR3, these TCRs were further manufactured in a TCR-T-IL12 lentiviral construct, where the expression of IL12 payload is regulated by TCR activation under a NFAT response element driven promoter (FIG. 6).

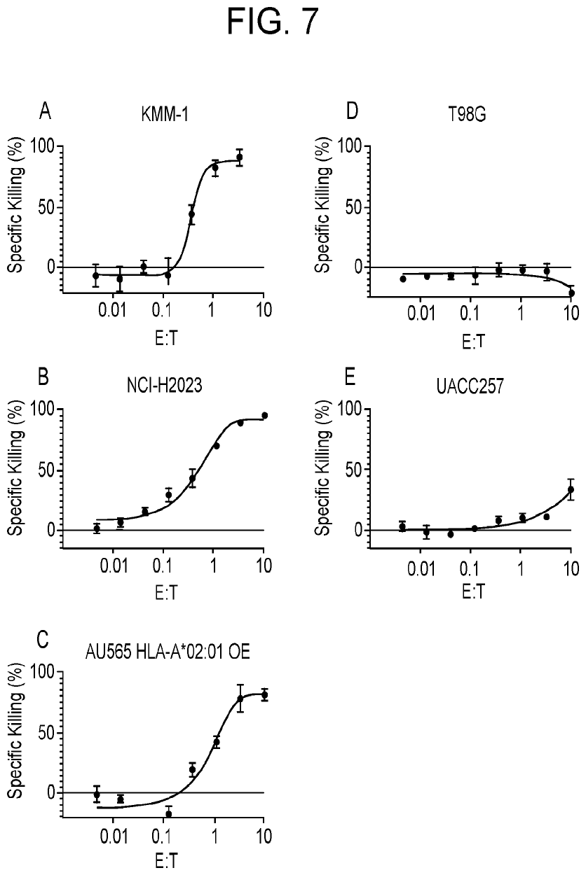

The potency of TCR2-IL12 T cells was subsequently evaluated in TDCC assays against multiple cancer cell lines. Consistent with previous assays with parental TCR2-T cells, TCR2-IL12 T cells demonstrated clear cytolytic activity against DCAF4L2+ HLA-A*02:01+ cancer cell lines such as KMM-1 and NCI-H2023, and HLA-A*02:01 overexpressing-AU565 (FIG. 7A-C), indicating TCR activity is not adversely affected by addition of the NFAT.IL12 construct. At the same time, TCR2-IL12 T cells displayed negligible cytolytic activity against DCAF4L2 negative cancer cells such as T98C and UACC257, when compared to an IL12-RFP control T cells (FIG. 7D-E), further confirming potency and specificity of the TCR2-IL12 T cells. DCAF4L2 mRNA transcript levels (FPKM) and DCAF4L2 target peptide presented by HLA-A*02:01 (copies per cell, cpc) quantified by mass spectrometry for each cancer cell line were shown below (Immatics). KMM-1: 7.91 FPKM, 124 cpc. NCI-H2023: 12.69 FPKM, 312 cpc. AU565 HLA-A*02:01 OE: 8.67 FPKM. T98C: 0 FPKM, Not detected cpc. UACC257: 0 FPKM, Not detected cpc.

### Example 3—Overview of Nonclinical Safety Assessment

An extensive in vitro and ex vivo safety assessment for TCR-T-IL12 cells was performed, as the human-specific HLA target precludes the use of animal models. First, target expression was assessed by various assays including transcriptome analysis (RNASeq) and mass spectrometry using normal human tissues as well as tumor tissues, as described above. Since DCAF4L2 is a cancer testis antigen, our studies displayed extremely restricted normal tissue expression (only expressed in testis). Second, off-target reactivity was assessed using two different strategies. The first strategy involved the evaluation of cytotoxicity against various human normal primary iPSC-derived cells types representative of major organs. The second strategy involved the identification of a panel of similar peptides based on sequence homology to the DCAF4L2 target peptide in conjunction with a positional scanning (X-Scan motif-based strategy to identify putative cross-reactive peptides unique to each TCR. To assess potential cross-reactivity to this panel of similar peptides, T2/peptide TDCC assays were conducted. The third safety assessment involved the evaluation of alloreactivity potential, using 34 B lymphoblastoid cell lines (BLCLs) expressing highly frequent HLA class I alleles in US populations, including 38 HLA-A, 40 HLA-B, and 24 HLA-C alleles.

### Identification of Similar Peptides Based on Homology

To assess off-target reactivity, a full panel of similar peptides to DCAF4L2 target peptide were identified using two different strategies, based on either sequence homology to target peptide or X-scan-derived motifs.

A homology-based strategy was designed to use an in-silico approach to identify a list of peptides that could potentially cross-react with the candidate TCR-Ts. To accomplish this, a protein database (UniProtKB/Swiss-Prot, June 2019) query was first performed to generate a list of all possible nonameric peptides, based on amino acid identity match to the target DCAF4L2 peptide (ILQDGQFLV). This in silico query was performed using a Python script and resulted in the identification of 150,046 peptides based on a 33.33% homology (identity) match to the target peptide. To refine this list further, criteria such as high homology match and software such as NetMHCpan and IEDB (The Immune Epitope Database) were utilized. NetMHCpan3.0 was used to consider a peptide's predicted binding affinity to HLA-A*02:01. IEDB database, which is a manually curated database of experimentally characterized immune epitopes, was used to consider a peptide's chance of being processed and presented by the HLA-A*02:01 allele. Specifically, the criteria used included the following (1) all peptides with greater than or equal to 66.67% homology match (identity) to the target peptide, which identified 35 peptides, (2) all peptides with greater than or equal to 55.56% homology match and predicted binding affinity (IC50) to be less than or equal to 50 nM, which identified 208 peptides, and (3) all peptides with greater than or equal to 55.56% homology match to target peptide that are reported in IEDB (presented by HLA-A*02:01 allele), which identified 20 peptides. As a result, this homology-based in silico search of the human proteome database led us to the identification of 243 unique peptides.

### Identification of the TCR Binding Motif Using Positional Scanning (X-Scan) and Similar Peptides Based on X-Scan-Derived Motifs

As an orthogonal approach to identify similar peptides, we used a positional scanning approach, known as X-scan. In X-scan, each residue of the DCAF4L2 peptide was sequentially mutated to one of other 19 naturally occurring amino acids, resulting in a total of 171 peptides. These 171 peptides were synthesized and tested in the T2/peptide TDCC assay to identify an X-scan derived motif that is specific to each individual TCR. Briefly, T2 cells were pulsed with each of these peptides at a 10 μM concentration, followed by addition of TCR-T cells at an E:T ratio of 1:1. Cell viability was determined using a T2/peptide TDCC assay. An amino acid substitution was defined as essential for TCR engagement where the viability observed was less than 20%. A corresponding search motif was constructed to express which amino acids were tolerated at each position in the peptide sequence (Table 4). Underlined amino acids in Table 4 represent the native residue at the corresponding position in the peptide. Using a python script, an in-silico search of the UniProtKB/Swiss-Prot database with splice variants was performed to identify all nonameric sequences that complied with the derived motif From this motif-based BLAST search, unique human peptide matches that conform to the consensus motif of the specific TCR-T were identified.

In the case of two TCRs (TCR2-T and TCR8-T), where the resulting motif search-based peptides were considerably large in number, further anchor residue restriction (at residues 2 and 9) was applied to the derived motif to limit final cross-reactive peptide selection (Table 1). Specifically, sequences of all 8763 nonameric HLA-A*02:01 positive peptides, obtained from the IEDB database (2019) and all 87 nonameric HLA-A*02:01 positive peptides obtained from PDB (Protein Data Bank, 2019) were analyzed to calculate the amino acid frequency at the anchor residue positions. A 1% amino acid frequency cut-off was applied to both the anchor residues (residue 2 and residue 9) of the motif, which restricted position 2to amino acids Y, S, E, T, A, Q, M, V, I, L, C and G and position 9 to amino acids T, M, F, Y, A, I, V, L, C and S.

TCR2-T was further tested in the X-scan experiment, using a peptide concentration of 100 nM, at an E:T ratio of 1:1. In this experiment, an amino acid substitution was defined as essential for TCR engagement where the viability observed was less than 50%. The corresponding search motif obtained from this experiment was also subjected to an in-silico search of the UniProtKB/Swiss-Prot database with splice variants to identify all nonameric sequences that complied with the derived motif. This led to the identification of 12 unique human peptides that were also further examined in the T2/peptide TDCC assay.

TABLE 4

| | | Unique peptide |
|---|---|---|
| | | matches, which |
| | | conform to the |
| TCRs | Motif obtained through Positional Scanning | consensus motif |
| TCR1-T | [IACEHLMPQSTVWY][LACDEFGHKNPQSTVWY][Q][DCN] | 7 |
| | [GS][QM][FIKW][LACGIMPQSTV][VACFIT] | |
| TCR2-T | [IACDEFHLMNPQSTVWY][LACEIMQSTVY][Q][DCN] | 50 |
| | [GCENSTW][QADMTVW][FTV][LAHIM][VACISTY] | |
| TCR3-T | [IACPTV][LCFIMTV][QCEIKLMNRV] | 29 |
| | [DN][GCE][QDE][FM][LCFHKMNQRSTY][VACEFILMQT] | |
| TCR8-T | [IACEFGHKLPQRSTVWY][LCMQSTY][QACHILMRV] | 52 |
| | [D][GE][QT][FACDHMPQV][LKV][VACILMST] | |
| TCR9-T | [IMV][LCFIMSTV][QCM][DCN][GE][QCDEGHNSTW][FY] | 16 |
| | [LACDEFGHIKMNPQRSTVWY][VACEFILMQT] | |

Cross-Reactivity Screen with Full Panel Similar Peptides

Full panel similar peptides (including the motif-based set and homology-based set) were synthesized and examined in T2/peptide TDCC assays to investigate the likelihood of off-target reactivity for each TCR-T.

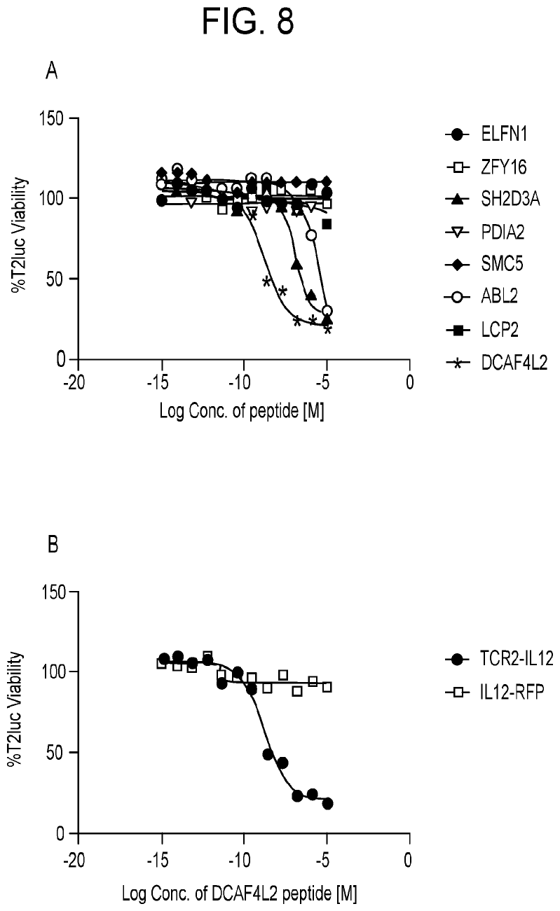

To identify potential cross-reactive peptides for each TCR-T-IL12, the full panel of similar peptides was tested using a T2/peptide TDCC screen with a high peptide concentration (10 μM). Peptides that showed less than 60% viability in a donor were considered as putative cross-reactive peptides and were selected for a further potency test. In case of TCR2-T-IL12, seven peptides were selected for a potency screen (FIG. 8A).

A potency gap of less than 10³-fold in EC50 in T2/peptide TDCC assays between target peptide and similar peptides was considered as a cutoff for further risk assessment. Cross-reactivity screen with full panel similar peptides for the TCR2-T-IL12 cells identified only a single putative cross-reactive peptide arising from SH2D3A (FIG. 8A). This nonameric peptide was identified through the X-Scan experiment and shared a 55.5% homology match to the antigenic DCAF4L2 peptide. The EC50 for putative cross-reactive peptide arising from SH2D3A was 87-fold as compared to the DCAF4L2 target peptide.

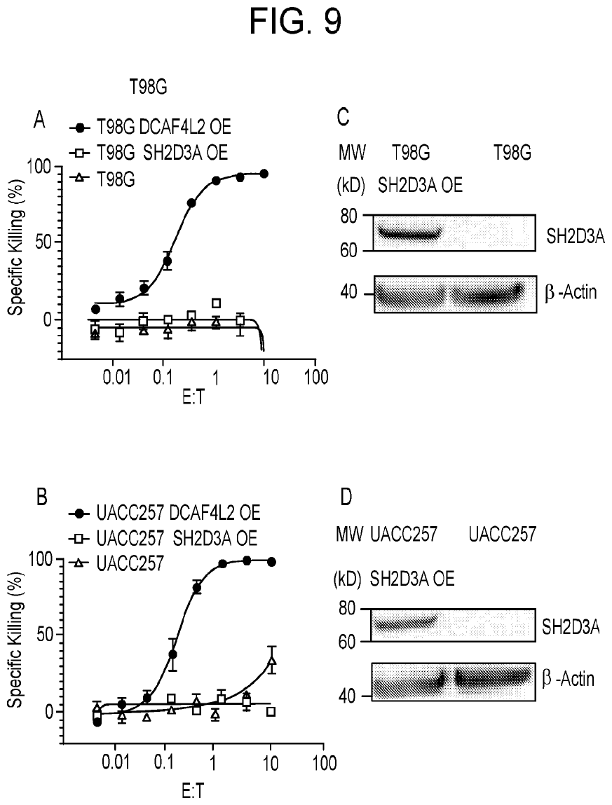

The identified putative cross-reactive peptide (SH2D3A) for TCR2-T-IL12 was further de-risked by TDCC assays with DCAF4L2 negative/HLA-A*02:01+ cancer cell lines (T98G and UACC257)-overexpressing the full length-protein (FIG. 9). Negligible cytolytic activities against both cancer cell lines overexpressing SH2D3A were observed by TCR2-T-IL12 (FIG. 9A-B), suggesting that this peptide is unlikely to be naturally processed and presented from the protein. Therefore, TCR2-IL 12 cross-reactivity against SH2D3A expressing HLA*02:01+ cells was inferred to be an unlikely safety concern.

In conclusion, TCR2-T-IL12 did not demonstrate any significant cross-reactivity across the full panel of similar peptides identified by sequence homology and X-scan-derived TCR motifs.

### Assessment of Cytotoxicity Against Human Normal Cells

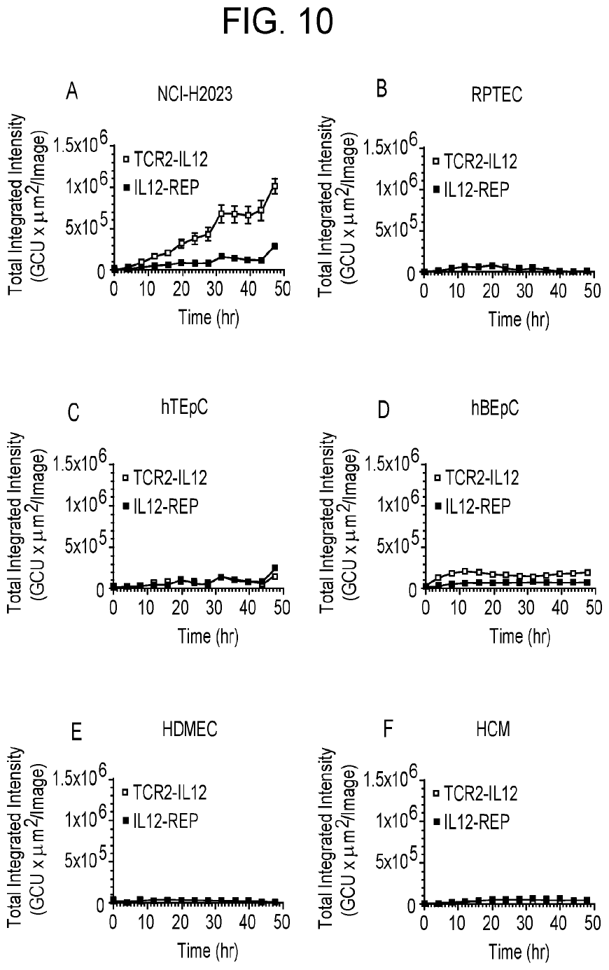

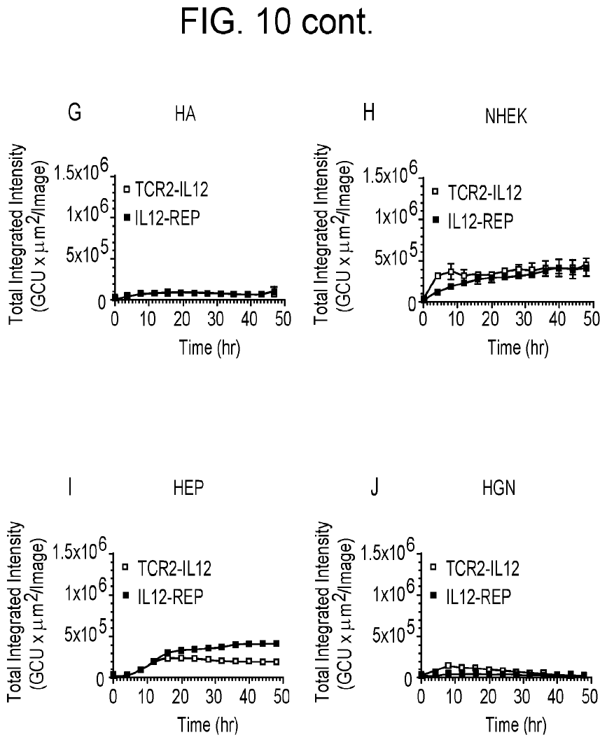

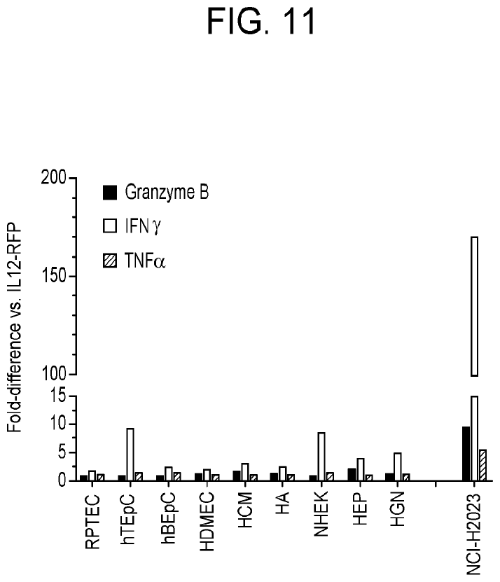

Next, the cytotoxicity of DCAF4L2 TCR2-IL12 T cells was evaluated against a panel of nine normal human primary or iPSC-derived cell types (without DCAF4L2 expression) representative of major organs serving as target cells in a TDCC assay. The nine human normal cell types including primary bronchial epithelial cells (hBEpC), tracheal epithelial cells (hTEpC), dermal microvascular endothelial cells (HDMEC), epidermal keratinocytes (NHEK), hepatocytes (HEP), renal proximal tubule epithelial cells (RPTEC), iPSC-derived astrocytes (HA). iPSC-derived cardiomyocytes (HCM), and iPSC-derived GABA neurons (HGN) (FIGS. 10 and 11). All human normal cells were obtained from HLA-A*02:01+ donors (HLA expression confirmed by RNASeq). Importantly, as these normal cells can present highly diverse peptides on HLA-A*02:01, this serves as an assay system to assess a broad range of off-target effects. The NCI-H2023 cancer cell line expressing both DCAF4L2 and HLA-A*02:01 was used as a positive control target cell. T cells expressing an IL12-RFP construct (but with no transgenic TCR) from the same donor as the TCR2-IL12 T cells, were included as negative control effector cells. Production of cytokines (IFNγ and TNFα) and granzyme B (FIG. 11), as well as target cell cytotoxicity (measured by caspase 3/7 cleavage) (FIG. 10) was assessed in co-culture of various human normal cell types with TCR2-T-IL12 cells. TCR2-IL12 T cells showed robust cytokine production and target cell cytotoxicity when co-cultured with the positive control NCI-H2023 cells (DCAF4L2+ HLA-A*02:01+) as expected. No strong caspase 3/7 activation was observed with any of the nine normal cell types tested, despite some low-level response with hBEpC and HGN. No increases greater than or equal to 3-fold (compared to the IL12-RFP control T cells) in granzyme B, or TNF-α responses against any of the nine normal cell types tested were observed. Increases in IFN-γ production greater than 3-fold (but less than 10-fold) were observed with selected cell types (hTEpC, HCM, NHEK, HEP and HGN). However, those cell types did not induce any other soluble analytes (Granzyme B, TNF-α and IL-12p70) assessed. Therefore, the lack of concomitant increases in multiple soluble mediators and/or caspase activation indicates the absence of significant safety concerns based on the normal cell reactivity assessment performed. Overall, TCR2-1L12 T cells did not show significant safety concerns based on the human normal cell cytotoxicity safety assessment performed.

### Assessment of Alloreactivity Using 34 BLCL Lines

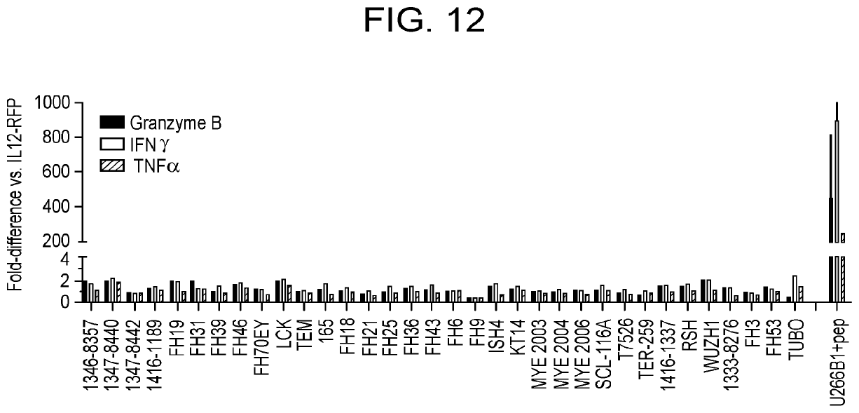

As part of the safety assessment, alloreactivity potential of the DCAF4L2 TCR2-IL12 was evaluated using a panel of 34 BLCLs representing frequent (≥11%) MHC Class I alleles in major US ethnic groups, including 38 HLA-A, 40 HLA-B, and 24 HLA-C alleles (Table 5). Alloreactivity potential was evaluated by the production of cytokines (IFNγ, TNFα, and IL-12p70) and granzyme B when TCR2-IL12 T cells were co-cultured with each of the BLCLs. No noteworthy increases in cytokine or granzyme B responses (greater than or equal to 3-fold compared to IL12-RFP control T cells) against the 34 BLCLs tested were observed for TCR2-IL 12 T cells (FIG. 12). However, TCR2-IL12 T cells demonstrated robust cytokine and granzyme B responses against positive control U266B1 cells (HLA-A*02:01±) pulsed with DCAF4L2 peptide (ILQDGQFLV).

Overall, TCR2-IL12 T cells did not show significant safety concerns based on the safety assessments performed to assess human normal cell cytotoxicity and alloreactivity potential.

### Example 4 Crystal Structure and Interaction Identification of the Dcaf4L2 Pmhc/Tcr2 Complex

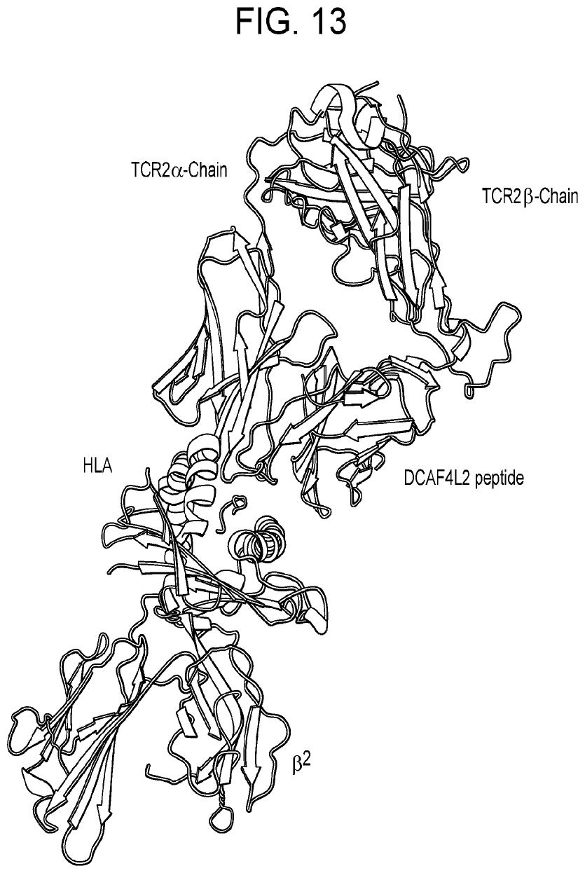

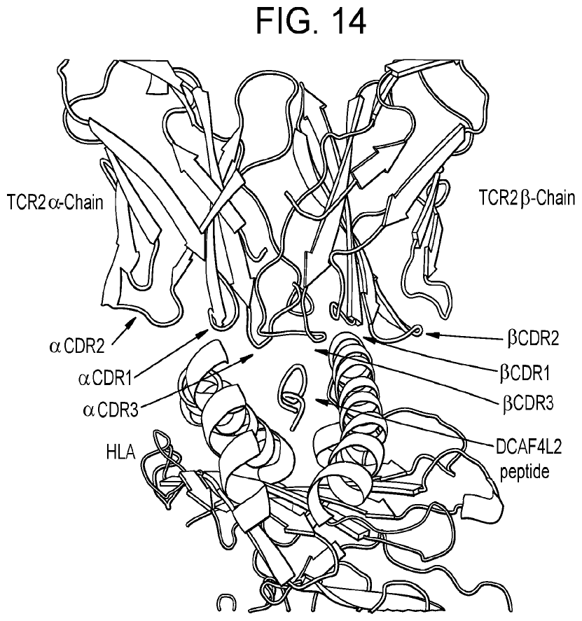

The DCAF4L2 peptide interacts with amino acid residues from the CDR1 & CDR3 loops of TCR2 and residues in the MHC a-helical binding cleft (FIGS. 13 and 14). Protein crystals of the DCAF4L2 pMHC/TCR2 complex were grown. The crystal structure of the DCAF4L2 pMHC bound to the TCR2 was determined at 2.8 Å resolution. The crystal structure shows that the TCR2 straddles the pMHC binding cleft, and CDRs 1 and 3 of both the a-chain and the P-chain of TCR2 are involved with the interaction with DCAF4L2 pMHC.

Specific core TCR2 amino acid residues of the interaction interface with DCAF4L2 peptide were defined as TCR2 residues that are within 5 Å of the DCAF4L2 peptide. The core residues are listed below.
α-chain: CDR1—R33
   CDR3—A94, W95, G96, Q100, G101
β-chain: CDR1—L32
   CDR2—Y52, R57
   CDR3—A97, G98, D99, R100, G101, Y102

Specific core DCAF4L2 peptide amino acid residues of the interaction interface with TCR2 were defined as DCAF4L2 peptide residues that are within 5 Å of the TCR2 protein. The core residues are listed below.
I1, Q3, D4, G5, Q6, F7, L8

Specific core DCAF4L2 peptide amino acid residues of the interaction interface with the HLA portion of the MHC were defined as DCAF4L2 peptide residues that are within 5 Å of the HLA protein. The core residues are listed below.
I1, L2, Q3, D4, G5, Q6, F7, L8, V9

Specific core MHC amino acid residues of the interaction interface with DCAF4L2 peptide were defined as MHC residues that are within 5 Å of the DCAF4L2 peptide. The core residues are listed below.
M5, Y7, F9, F33, M45, Y59. E63, K66, V67, H70, T73, V76, D77, T80, L81, Y84, R97, Y99, H114, Y116, Y123, T143, K146, W147, A150, V152, Q155, L156, Y159, T163, W167, Y171

Specific core MHC amino acid residues of the interaction interface with TCR2 were defined as MHC residues that are within 5 Å of the TCR2 protein. The core residues are listed below.
E58, R65, K66, A69, Q72, T73, R75, V76, W147, A150, H151, E154, Q155, L156, A158, Y159, T163

In conclusion, analysis of the crystal structure allowed for identification of specific amino acids involved in the interaction between the DCAF4L2 peptide, TCR2 and the MHC, the core regions of the interface on each protein, and the spatial requirements of the TCR2 to interact with the DCAF4L2 pMHC.

### Methods and Materials Used in the Above Examples

DCAF4L2 pMHC-Specific TCR Identification by Healthy Donor Screen

### Generation of Autologous Antigen Presenting Cells (APCs)

HLA-A*02:01 positive healthy donor peripheral blood mononuclear cells (PBMCs) were obtained from both frozen and fresh AllCells or PPA. Monocytes were positively selected from PBMCs by using human CD14-microbeads (Miltenyi Biotec, San Diego, CA, 130-050-201). Mature dendritic cells were obtained by using CellXVivo™ Human Monocyte-derived Dendritic Cell (DC) Differentiation Kit (R&D, Minneapolis, MN, CDK004). Antigen-presenting B cells were generated by using CD40L and IL-4 stimulation method. B cells were positively selected by using human CD19-microbeads (Miltenyi Biotec, 130-050-301) from PBMCs. CD19+ cells were then stimulated by 0.125 ug/ml recombinant huCD40L in B cell media and seeded in 24-well plate at 2×10⁵ cells/ml and 1 ml/well. B-cell media comprised of IMDM, GlutaMax™ supplement media (Gibco, 31980030) supplemented with 10% heat inactivated human serum (Millipore Sigma H3667-100ML), 100 U/ml penicillin and 100 μg/ml streptomycin (Gibco, 15140-122), 10 μg/ml gentamicin (Gibco, 15750-060) and 200 IU/ml IL-4 (Peprotech, 20004100UG). Fresh B cell media with 400 IU/ml IL-4 was added to the B cell culture at 1 ml/well on day 3 post B cell activation without disturbing the cells. Activated B cells were ready to use for antigen-reactive T cell stimulation on day 6 post B cell activation.

### Ex Vivo Stimulation and Expansion of Antigen-Specific T Cells

DCAF4L2 peptide (Anaspec customized peptide, Freemont, CA) was added to the immature dendritic cells at 1 μM along with recombinant human TNFα on day 7 post CD14+ cell isolation. On day 9 post CD14+ cell isolation, DCAF4L2 peptide-pulsed mature dendritic cells were collected, washed, and mixed with CD14− PBMCs at ratio I to 10 in human T cell media with 10 μM DCAF4L2 peptide, 10 IU/ml IL-2 (Miltenyi Biotec, 130-097-745) and 10 ng/ml IL-7 (Peprotech, AF20007100UG). Human T cell complete media consists of a 1 to 1 mixture of CM and AIM-Val (Thermo Fisher, 12055083). CM consists of RPMI 1640 supplemented with GlutaMAX™ (Gibco, 61870-036), 10% human serum (MilliporeSigma, H3667), 25 mM HEPES (Gibco, 15630-080) and 10 μg/ml gentamicin (Gibco, 15750-060). DCAF4L2 specific T cells were further expanded by one to four rounds of weekly peptide-pulsed B cell activation. HuCD40L activated B cells were collected, washed, and seeded in 6-well plate at 1×10⁶ cells/ml and 4 ml/well, 1 μM DCAF4L2 peptide was added to the B cells and incubated at 37° C. for 2 hours in the incubator. The peptide-pulsed B cells were then mixed with the T cells at a ratio of 1:10 in human T cell media with 10 IU/ml IL-2 and 10 ng/ml IL-7. DCAF4L2 dextramer positive cells were confirmed by flow cytometry and then sorted for TCR identification by single cell RNAseq.

### Sorting of Activated Antigen-Specific T Cells

DCAF4L2 peptide activated antigen-specific T cells were stained with DCAF4L2 dextramer-APC and -PE at room temperature in dark for 10 min and then stained by CD3-FITC (Biolegend, 300440) and CD8-BV605 (BD Biosciences, 564116). The dead cell exclusion stain (Sytox blue) was purchased from ThermoFisher (Invitrogen, S34857). Cells were sorted using an Ariam Fusion cell sorter (BD Biosciences, San Jose, CA). Data were analyzed using Flowjo post-sort.

### Elispot

The sorted CD3+CD8+Dex+ T cells were validated for the antigen-specific IFNγ production by BD® ELISPOT assay (BD, 551849) using peptide-loaded T2 cells. T2 cells were loaded with 10 M DCAF4L2 peptide in human T cell complete media at 2×10⁶ cells/ml and 1 ml/well in 24 well plate for 1-2 hours. 150 ul of human T cell complete media and 50 μl of peptide-loaded T2 cells were added to each well in the pre-coated ELISPOT plate. The CD3+CD8+Dex+ T cells (500 or 1000 cells) were directly sorted into each well in the ELISPOPT plate. The ELISPOT was detected after 24-hour incubation in 37° C. incubator. The ELISPOT plates were scanned and counted by IMMUNOSPOT® (Cellular Technology Limited, Cleveland, OH).

### Single Cell RNAseq

Samples were processed using a Chromium™ Controller (10× Genomics, Pleasanton, CA) with the V(D)J single-cell Human T Cell enrichment kit (PN-1000006, PN-1000005, PN-120236, PN-120262) according to manufacturer's instructions for direct target enrichment, skipping cDNA amplification step for the full transcriptome. Briefly, cells and beads with barcoded oligonucleotides were encapsulated in nanoliter droplets where the cells were lysed, and mRNA reverse transcribed with poly-T primers and barcoded template-switch oligos. Nested PCR was then performed with primers in the constant region of the human TCR and template-switch oligo. The second target enrichment PCR was performed using 13-17 cycles depending on estimated cell input number according to manufacturer's suggestions. The final sequencing library was generated from fragmented PCR product ligated to Illumina sequencing adapters. Libraries were sequenced with 151 paired end reads (151×8×0×151) on NextSeg™ 550 or MiSeg™ (Illumina, Inc., San Diego, CA) at a depth of at least 5,000 reads per cell. Data was demultiplexed and analyzed with cellranger vdj (2.2.0) to obtain full-length paired TCR sequences assigned to individual cells.

Cloning and Transduction of TCRs into Jurkat Cells

Candidate TCRs were generated as gene fragments. Each fragment was cloned into a lentiviral expression vector consisting of a MSCV promoter and an IRES-driven eGFP for monitoring transfection or transduction. Successful transformants were screened by Sanger sequencing and verified clones were maxi-prepped for downstream applications. In those cases where transduction was used to screen a candidate TCR, the lentiviral vector was packaged into VSV-G pseudotyped virions (Alstem, Richmond, VA). Lentivirus carrying TCRs were transduced into a Jurkat TCR KO reporter cell line expressing CD8a constitutively and Renilla luciferase under a NFAT inducible promoter. Briefly, 20 μL of lentivirus particles were added to between 100K and 1 million cells in complete media containing 5 μg/mL Polybrene (MilliporeSigma, TR1003G) in a 50 mL conical tube such that the multiplicity of infection (MOI) was 10. After the addition of virus, cells were spun at 1200×g for 45 min at 32° C. After the spin, the media was aspirated and replaced with sufficient fresh media to adjust the cells to a concentration of 500K cells/ml before being placed in a 37° C. incubator. Approximately 72 hours post-transduction, cells were analyzed by flow cytometry. 501 of cells were transferred to a 96-well U-bottom plate and 150 μl FACS buffer (PBS w/o CaCl₂ & MgCl₂ (Corning, 21-040-CV)+5% FBS (Gibco, 10082-147)) was added before being centrifuged at 300×g for 3 min. Supernatant was removed and cells were resuspended in 50 μl of 1× Fc block in FACS buffer which was incubated at 4° C. for 20 min. Fluorescent dextramer specific to DCAF4L2 peptide-MHC (ILQDGQFLV/HLA-A*02:01, Immudex customized) was incubated with transduced cells at room temperature for 10 min in the dark using the manufacturer's recommended concentration. Afterward, a 2× antibody cocktail containing anti-CD3 (BD Biosciences) in 50 ul volume was added before another incubation at 4° C. for 20 min. Cells were washed three times after staining by centrifugation at 300×g for 3 min followed by aspiration and resuspension. Prior to analysis, cells were fixed in 100 μl of fresh 2% formaldehyde solution at 4° C. for 20 min. Cells were washed twice to remove the formaldehyde before final suspension in 200 μl of PBS with EDTA. Fixed, labeled cells were run on either LSRII or Symphony™ cytometers (BD Biosciences) using recommended acquisition settings.

### Jurkat Activation Assay

Antigen-presenting T2 cells (ATCC) were loaded with peptides (Anaspec customized) or vehicle only at a range of concentrations in serum-free media for two hours. After incubation, loaded T2 cells were washed three times before being resuspended in complete media, counted, and seeded at 15,000 cells/well in a half area 96 well plate (Corning). Successfully transduced Jurkat cells were added at 30,000 cells/well to a total volume of 100RL. The TCR-expressing Jurkat cells were co-cultured at 37° C. in the presence of the T2 cells for 24 hours. At the end of this incubation, the plate was briefly centrifuged at 300×g before half the volume was harvested and stored for characterization of cytokine secretion. To the remaining volume was added an equal volume of RENILLAGLO® (Promega) and the plate was incubated for 20 min at room temperature with shaking before luminescence was detected on an ENVISION® (Perkin Elmer, Waltham, MA). The activities of individual TCRs were expressed as the fold change of the luminescence in the presence of T2 cells loaded with peptide compared to co-cultures with vehicle-only T2 cells.

DCAF4L2 TCR-T and TCR-T-IL12 cell production using human primary T cells PBMCs from three healthy donors (HLA-A*02:01) were isolated from leukopak (Allcells) using Ficoll-Paque gradient centrifugation, with additional T cell isolation by using CD3 negative selection kit (Miltenyi Biotec, 130-096-535) and associated manufacturer's protocol. One day before TCR transduction, frozen pan-T cells were thawed and resuspended in Human T cell complete media at 1×10⁶ cells/ml, and were stimulated by CD3/CD28 Dynabeads™ (Thermo Fisher, 111311D) with T cells to beads ratio (2:1) in the presence of 30 IU/ml IL-2 (Miltenyi Biotec, 130-097-745), 10 ng/ml IL-7 (Peprotech, AF20007100UG) and 25 ng/ml IL-15 (Peprotech, AF20015100UG). The T cells were then seeded at 1 ml per well in 24-well plates. On the day of TCR transduction, activated T cells (300K) were seeded in Human T cell complete media per well in 48-well plate and transduced with lentivirus in the presence of 8 μg/ml polybrene, 100 IU/ml IL-2, 10 ng/ml IL-7, and 25 ng/ml IL-15. The T cells were then spin-inoculated at 1500×g for 1.5 hours at 32° C. After spin-inoculation, 380 ul of media with 8 μg/ml polybrene, 100 IU/ml IL-2, 10 ng/ml IL-7, and 25 ng/ml IL-15 was added to the cells to make a total volume of 600 μl per well. At 17-18 hours post transduction, ˜400 μl of media was removed without touching the cells at the bottom of the wells. The cells from each well of 48-well plate were transferred to one well of G-REX® 24-well plate (WilsonWolf, P/N 80192M) in 3 ml of Human T cell complete media containing 100IU/ml IL-2, 10 ng/ml IL-7, and 25 ng/ml IL-15. On day 4 post transduction, the Dynabeads were removed according to manufacturer's protocol. The TCR-T cells were seeded to G-REX® 6-well plate (WilsonWolf, P/N 80240M) at ˜10×10⁶ cells in 30 ml media per well in the presence of 100IU/ml IL-2, 10 ng/ml IL-7, and 25 ng/ml IL-15. On day 7 post transduction, the TCR-Ts were harvested, frozen down, and stored in liquid nitrogen vapor phase. TCR transduction efficiency was validated by dextramer binding. The TCR-T-IL12 cells were produced by the process described in the patent application (PCT published application number: WO 2021211104).

### Flow Cytometry

The following antibodies were used for T cell phenotyping: CD3-FITC (Biolegend: 300440). CD8-BV605 (BD: 564116), CD4-PE (Biolegend: 317410). The following antibodies were used for dendritic cell phenotyping: CD14-PerCP/Cy5.5 (Biolegend: 301824), CD11c-PE (Biolegend: 337206), CD1a-APC-cy7 (Biolegend: 300125), CD86-APC (BD: 555660). The following antibodies were used for B cell phenotyping: MHC class I (Biolegend: 311414), MHC class II (Biolegend: 361706), CD83-PE (BD 556855), CD86-APC (BD: 555660), CD20-FITC (BD: 556632). Dextramers-APC or -PE were purchased from Immudex (customized dextramers). 50 nM PKI dasatinib (Axon Medchem: 1392) was used to prevent TCR internalization. The TCR expressing T cells were incubated with 50 nM PKI dasatinib at 37° C. for 30 min and then followed by dextramer staining on ice for 30 min and cell surface marker staining at 4° C. for 15 min, The dead cell exclusion stain (Sytox blue, ThermoFisher/Invitrogen, S34857) was used. Flow cytometry data were analyzed using Flowjo.

### T Cell-Mediated T2-Luc/Peptide Cytotoxicity Assay (T2/Peptide TDCC Assay)

Functionality and killing specificity of DCAF4L2 TCR-T was determined by T2-luc (T2 cell line expressing luciferase) killing assays. T2-luc cells were collected, washed and resuspended at 1×10⁶ cells/ml in killing assay media (RPMI 1640—GlutaMAX™, 1× Non-Essential Amino Acids Solution (Gibco, 11140-050), 10 mM HEPES (Gibco, 15630-080), 50 μM 2-B-mercaptoethanol (Gibco, 21985-023), 1 mM sodium pyruvate (Gibco, 11360-070), 100 U/ml Penicillin-Streptomycin (Gibco, 15140-122), 5% heat-inactivated FBS (Gibco, 10082-147)), and then seeded at 1 ml per well in 24-well plate. T2-luc cells were pulsed with the indicated peptide concentrations for two hours at 37° C. T2-luc cells were then washed and resuspended at 1×10⁵ cells/ml in killing assay media and seeded at 25 μl per well in 384-well plates (Corning, 350).

Previously frozen donor TCR-Ts were thawed, washed, and resuspended in warm killing assay media prior to use in assays. The number of TCR-T cells per well was normalized for equivalent number of DCAF4L2 dextramer+ cells and total number of cells. TCR-T cells for each donor were normalized to the lowest frequency DCAF4L2 dextramer+ TCR-T prior to being added to co-culture assay. Total cell number was equalized among TCR-Ts with the addition of mock transduced donor T cells.

For E:T titration assays, T2-luc cells were incubated with TCR-T cells at the indicated dextramer+TCR-T cell to T2-luc cell ratios. For DCAF4L2 peptide titration assays, E:T ratio was fixed at 1:1. After 72 hours, the luminescent signal was measured by addition of 30 μl of SteadyGlo (Promega, E2520) followed by measurement of luminescent signals by using Biostack™ neo system (BioTek, Winooski, VT). For E:T titration assays, specific lysis was calculated through normalization of TCR-T+T2/target peptide killing to mock T cells+T2/target peptide killing for each E:T ratio. For DCAF4L2 peptide titration assays, specific lysis was calculated through normalization of TCR-T+T2/target peptide killing to TCR-T+ unpulsed T2 cells for each TCR-T. Specific lysis formulas are described below.

To evaluate the ability of additional HLA-A*02 alleles to present target peptide to TCR-T cells, a T2-luc double knockout cell clone (CD3ε KO/HLA-A*02:01 KO) was generated and referred to as D5 cell line. D5 cell line was transduced with MSCV retrovirus carrying different HLA-A*02 alleles including HLA-A*02:01, HLA-A*02:03, HLA-A*02:05, HLA-A*02:06, or HLA-A*02:07. In peptide titration assays, E:T ratio was fixed at 1:1. After 72 hours, the luminescent signal was measured by addition of 30 μl of Bio-Glo (Promega, G7940) followed by measurement of luminescent signals by using Biostack neo system. For evaluation of TCR-Ts against HLA-A2 family alleles expressed on D5 cells in a DCAF4L2 peptide titration assay, specific lysis was calculated through normalization of TCR-T+T2/target peptide killing to mock (untransduced) T cells+T2/target peptide killing for each peptide concentration.

### Formula for Specific Lysis (%)

### Peptide Titration (DCAF4L2 Peptides and Similar Peptides):

{1−(TCRT+T2−luc/test peptide RLU)/(TCRT+T2−luc/no peptide RLU)}×100

E:T titration and peptide titration on HLA-A2 allele family-D5 cell lines (DCAF4L2 peptide):

{1−(TCRT+T2−luc/DCAF4L2 peptide RLU)/(Mock TCRT+T2−luc/DCAF4L2 peptide RLU)}×100

### Cancer Cell Line Killing:

{1−(TCRT+cancer cell line RLU)/(MockT+cancer cell line RLU)}×100

### T Cell-Mediated Cancer Cell Cytotoxicity Assay (Cancer Cell TDCC Assay)

Cytotoxicity of TCR-T cells against DCAF4L2 positive and negative cancer cell lines was determined by cancer cell killing assay. Cancer cells were collected, washed and resuspended at 1×105 cells/ml in cancer cell killing assay media (RPMI 1640—GlutaMAX™, 1× Non-Essential Amino Acids Solution (Gibco, 11140-050), 10 mM HEPES (Gibco, 15630-080), 50 μM 2-ß-mercaptoethanol (Gibco, 21985-023), 1 mM sodium pyruvate (Gibco, 11360-070), 100 U/ml Penicillin-Streptomycin (Gibco, 15140-122), 5% heat-inactivated FBS (Gibco, 10082-147)).

Cancer cells were then seeded at 25 μl per well in 384-well plates and incubated with 25 μl of TCR-T cells with the indicated dextramer+TCR-T to T2-luc cells ratio for 72 hours. Following incubation, for adherent cancer cells, the suspension T cells were removed by washing with DPBS with Ca²⁺Mg²⁺ (Corning, 21-031-CM) using a plate washer. The luminescent signal was measured by addition of 30 μl of Celltiter Glo (Promega, G7573). For suspension luciferase-labeled cancer cells, the luminescent signal was measured by addition of 30 μl of Bio-Glo™ (Promega, G7940). Biostack neo system was used for luminescence measurement. Signal was normalized against cancer cells co-cultured with relevant empty, mock, or IL12-RFP T cell controls. Specific lysis formula is described above.

To generate DCAF4L2 or SH2D3A full length protein overexpressing cell lines, coding gene fragments (IDT DNA Technologies) were cloned into plasmid under an EF apromoter using an In-Fusion HD Cloning Plus Kit (Takara Bio) with a T2A-EGFP reporter sequence. Successful transformants were screened by Sanger sequencing and verified clones were maxi-prepped for downstream applications. The plasmid was packaged into lentiviral vectors, which were subsequently used to transduced T98G and UACC257 cells through spinfection for 1.5 h at 1500×g. Successfully transduced cells were sorted via FACS before use in cancer cell killing assays as described above.

### Similar Peptide Screen

Functional specificity of DCAF4L2 TCR-T was determined using T2-Luc/peptide directed killing assays. Peptides including target and similar peptides were synthesized by JPT (Berlin, Germany) or AnaSpec (Fremont, CA). T2-Luc cells were incubated with reactive similar peptides, target specific peptide or DMSO control in T2-Luc killing media at a final peptide concentration range of 1.0E-05M to 6.0E-16M (potency) or 1.0E-05M (single point) for 2 hours at 37° C./5% CO₂. Frozen DCAf4L2 TCR-T and IL12-RFP cells were thawed, washed, and rested in human T cell media for 3 hrs prior to assay set-up. DCAF4L2 TCR-T cells were washed 3× in assay media and re-suspended at 2.5E06 cells/mL. Peptide loaded T2-Luc cells were added to white-clear bottom 384-well assay plates (Costar) at 2,000 cells/25 μL using Bravo liquid handling system (Agilent, Santa Clara, CA). DCAF4L2 TCR-T cells were prepared by diluting DCAF4L2 dextramer positive cells with mock T-cells to obtain a 10:1 target: effector ratio; 20,000 cells/25 μL (final 1:1 Dex+ T cell: T2-Luc). T2-Luc pulsed cells and TCR-T cells were incubated for 48 hours at 37° C./5% CO2. T2-Luc cell viability was determined using Bio-Glom Luciferase Assay System (Promega, G7940) according to the manufacturer's recommendation. Luminescence was detected using ENVISION® Multilabel Plate Reader (Perkin Elmer, Santa Clara, CA). Percent viability was calculated using the following formula: % Viability=(Sample raw RLU value/Average DMSO control RLU)×100. EC50 was determined using GraphPad Prism (non-linear regression curve fit analysis).

### Human Normal Cell Culture

Sources of human primary normal cells and iPSC-derived cells are summarized in Table 6. Culture conditions for these cells are summarized in Table 7. Primary cells were thawed and maintained according to the supplier's instructions prior to co-culture with TCR-T-IL12 cells.

TABLE 6
Source of human normal primary and iPSC-derived cells

| | Cell | | | |
|---|---|---|---|---|
| Cells | Type | Source | Donor | Catalog # |
| Bronchial | Primary | PromoCell, | 424Z015.3 | C-12640 |
| Epithelial Cells | | Heidelberg, | | |
| (hBEpC) | | Germany | | |
| Renal Proximal | Primary | Lonza, Basel, | 682573 | CC-2553 |
| Tubule Epithelial | | Switzerland | | |
| Cells (RPTEC) | | | | |
| Tracheal | Primary | PromoCell | 446Z036.8 | C-12644 |
| Epithelial | | | | |
| Cells (hTEpC) | | | | |
| Epidermal | Primary | PromoCell | 453Z017.1 | C-12003 |
| Keratinocytes | | | | |
| (NHEK) | | | | |
| Dermal | Primary | PromoCell | 435Z034.2 | C-12212 |
| Microvascular | | | | |
| Endothelial | | | | |
| Cells (HDMEC) | | | | |
| Hepatocytes | Primary | Lonza | HUM17299A | HUCPG |
| (HEP) | | | | |
| GABA Neurons | iPSC | Cellular | 01434 | R1012 |
| (HGN) | | Dynamics, | | |
| | | Madison, WI | | |
| Astrocytes | iPSC | Cellular | 01434 | ASC-100- |
| (HA) | | Dynamics | | 020-001- |
| | | | | PT |
| Cardiomyocytes | iPSC | Cellular | 01434 | R1006 |
| (HCM) | | Dynamics | | |

TABLE 7
Culture media and methods for human normal cells

| | | | | Plating |
|---|---|---|---|---|
| | | Maintenance | | Density |
| Cells | Assay Medium | medium | Specific Methods | (cells/well) |
| Bronchial | 1:1 mixture of | Airway | Plated normal cells directly | 10,000 |
| Epithelial Cells | complete RPMI | Epithelial | into 96-well flat-bottom plate | |
| (hBEpC) | medium and | Cell Medium | in maintenance medium and | |
| | Airway | | mix with TCR2-IL12 cells in | |
| | Epithelial Cell | | equal volume of complete | |
| | Medium | | RPMI. | |
| Renal Proximal | 1:1 mixture of | Renal | Plated normal cells directly | 10,000 |
| Tubule Epithelial | complete RPMI | Epithelial | into 96-well flat-bottom plate | |
| Cells (RPTEC) | medium and | Cell Growth | in maintenance medium and | |
| | Renal Epithelial | Medium | mix with TCR2-IL12 cells in | |
| | Cell Growth | | equal volume of complete | |
| | Medium | | RPMI. | |
| Tracheal | 1:1 mixture of | Airway | Plated normal cells directly | 10,000 |
| Epithelial Cells | complete RPMI | Epithelial | into 96-well flat-bottom plate | |
| (hTEpC) | medium and | Cell Medium | in maintenance medium and | |
| | Airway | | mix with TCR2-IL12 cells in | |
| | Epithelial Cell | | equal volume of complete | |
| | Medium | | RPMI. | |
| Epidermal | 1:1 mixture of | Keratinocyte | Plated normal cells directly | 10,000 |
| Keratinocytes | complete RPMI | Growth | into 96-well flat-bottom plate | |
| (NHEK) | medium and | Medium 3 | in maintenance medium and | |
| | Keratinocyte | | mix with TCR2-IL12 cells in | |
| | Growth | | equal volume of complete | |
| | Medium 3 | | RPMI. | |
| Dermal | 1:1 mixture of | Endothelial | Plated normal cells directly | 10,000 |
| Microvascular | complete RPMI | Cell Growth | into 96-well flat-bottom plate | |
| Endothelial Cells | medium and | Medium | in maintenance medium and | |
| (HDMEC) | Endothelial Cell | | mix with TCR2-IL-12 cells | |
| | Growth | | in equal volume of complete | |
| | Medium | | RPMI. | |
| Hepatocytes | 1:1 mixture of | Hepatocyte | Thawed in Hepatocyte | 10,000 |
| (HEP) | complete RPMI | Maintenance | Thawing Medium; plated in | |
| | medium and | Medium | Hepatocyte Plating Medium | |
| | Hepatocyte | | into collagen-coated 96-well | |
| | Maintenance | | ViewPlates; after 4-6 hr | |
| | Medium | | incubation, cells were | |
| | | | washed and maintained in | |
| | | | Hepatocyte Maintenance | |
| | | | Medium before mixing with | |
| | | | TCR2-IL12 cells in equal | |
| | | | volume of complete RPMI. | |
| GABA Neurons | 1:1 mixture of | iCell Neural | Plated directly in Neural | 10,000 |
| | complete RPMI | Medium | Medium into 96-well flat- | |
| | medium and | | bottom plate coated | |
| | iCell Neural | | sequentially with 50 μg/mL | |
| | Medium | | PDL and 3.3 μg/mL laminin. | |
| | | | After 24 hr incubation, cells | |
| | | | were washed and maintained | |
| | | | in fresh Neural Medium | |
| | | | before mixing with TCR2- | |
| | | | IL12 cells in equal volume of | |
| | | | complete RPMI. | |
| Astrocytes | 1:1 mixture of | DMEM/F12 | Plated directly in | 10,000 |
| | complete RPMI | with 2% FBS | DMEM/F12 with 2% FBS | |
| | medium and | and 1X N-2 | and 1X N-2 Supplements into | |
| | DMEM/F12 | supplement | 96-well flat-bottom plate | |
| | with supplements | | coated with 10 μg/mL | |
| | | | laminin. After 24 hr | |
| | | | incubation, cells were | |
| | | | washed and maintained in | |
| | | | DMEM/F12 with supplements | |
| | | | before mixing with | |
| | | | TCR2-IL12 cells in | |
| | | | equal volume of | |
| | | | complete RPMI. | |
| Cardiomyocytes | 1:1 mixture of | HCM | Plated directly in HCM | 10,000 |
| | complete RPMI | Maintenance | Plating medium into 96-well | |
| | medium and HCM | Medium | flat-bottom plates coated with | |
| | Maintenance | | 0.1% gelatin. After 48 hr | |
| | Medium | | incubation, cells were | |
| | | | washed and maintained in | |
| | | | HCM Maintenance Medium | |
| | | | before mixing with TCR2- | |
| | | | IL12 cells in equal volume of | |
| | | | complete RPMI. | |

Cytotoxicity Assays with Human Primary Normal Cells

Target cell cytotoxicity was assessed using a phase contrast/fluorescence kinetic imaging assay. Fluorescent caspase 3/7 cleavage was measured over time with an INCYCYTE® (Sartorium, Gottingen, Germany) live imaging device and overlaid onto phase contrast images that captured cell confluence. Prior to implementing the cytotoxicity assay, different plating densities and tolerability to various culture media were assessed to achieve suitable confluence without significant cell overlap in 96-well plates. 10,000 target cells (100 L) per well were plated in 96-well plates and co-cultured with 100 μL of DCAF4L2 TCR2-IL12 cells or IL12-RFP T cells at an effector: target (E:T) ratio of 1:1 and 2:1 respectively in order to keep the total number of T cells consistent reflective of the different transduction efficiencies for TCR2-IL12 (22.9%) and IL12-RFP T cells (47.7%). CellEvent™ caspase 3/7 reagent was added to final concentration of 5 μM according to the manufacturer's instructions (ThermoFisher, C10423). Assay plates were placed in a 37° C., 5% CO₂ incubator equipped with an INCUCYTE® S3. Phase contrast and fluorescent images (5 fields) with the 10× objective were collected every 4 hours starting at 0 hour for 48 hours and analyzed for Caspase 3/7 total integrated intensity using IncuCyte® 2020B software. A minimum area filter was set at 200 μm² in fluorescent images to exclude signals from apoptotic T cells. In addition, since fluorescent signals in target cells were not homogeneous, target cells could be recognized as smaller splits and excluded by area filter. Therefore, edge detection was turned off during analysis. After 48 hours, plates were removed from the incubator, centrifuged at 400×g for 5 minutes and 50 μL of cell culture medium was removed from the wells for cytokine analysis.

Cytokine Assay with Human Primary Normal Cells

Cell culture supernatants (50 μL) were collected from cytotoxicity assays at 48 hours into 96-well plates. Cytokines and Granzyme B were evaluated by Luminex assay using a custom MILLIPLEX® Human Cytokine/Chemokine Kit (Millipore, SRP1885), including the analytes of IFNγ, granzyme B and TNFα, as per manufacturer instructions. Serial dilutions of analyte standards were run in replicates on each assay plate. The Luminex plate was read on a FLEXMAP 3D® instrument (XMAP® technologies). Data was exported by XPONENT® Software and analyzed directly by EMD Millipore's MILLIPLEX® Analyst software, generating standard curves using a 5-parameter logistic non-linear regression fitting curve. The limits of detection (Min and Max) were calculated by the MILLIPLEX® Analyst software as the result of the average of appropriate replicate standard curve values obtained from each assay plate and indicate the range within which an analyte can be interpolated from the standards. Samples were run at appropriate dilutions to ensure measurements of sample analyte levels were within assay standard curve limits. Cytokine and granzyme B levels are reported in pg/mL or as fold-differences over IL12-RFP T cells (controls) and graphed in GraphPad Prism software.

### Alloreactivity Screen

Alloreactivity potential was assessed by co-culture of TCR2-IL12 T cells with each of 34 BLCLs representing 38 HLA-A, 40 HLA-B, and 24 HLA-C alleles. BLCLs were purchased from Fred Hutchinson Cancer Research Institute (“Fred Hutch”; Seattle, WA) and Astarte Biologics (Cellero; Bothell, WA) as listed in Table 5. BLCLs were cultured in 15% FBS complete RPMI containing: RPMI-1640 with L-Glutamine, 15% (v/v) HI-FBS, and 1 mM Sodium Pyruvate.

U266B1 cells (ATCC; 10⁵ cells/ml in media), as an HLA-A*02:01⁺ positive control cell line, were pulsed with 50 μM DCAF4L2 peptide (ILQDGQFLV) by incubation at 37° C. for 2 hours. TCR-T cells from donor D110048238 were thawed by addition of media, centrifuged at 400×g for 5 min at 4° C., resuspended in 10 ml of media, and counted. 2.183×10⁵ TCR-T cells were co-cultured with either 1×10⁴ BLCLs or DCAF4L2 peptide-pulsed U266B1 cells in 200 μl volume. The dextramer-normalized effector: target ratio for TCR2-IL12 was 5:1, and for IL12-RFP control T cells was 10:1, in order to keep the total number of T cells consistent between the TCR2-IL 12 and IL 12-RFP assay wells. All co-cultures were conducted in 96-well flat-bottom tissue culture plates at 37° C., 5% C02 for 48 hours. Following incubation, the 96-well plates were centrifuged at 887×g for 1 min at 4° C. and the supernatant was collected into 96-well V-bottom plates for cytokine analysis. Cytokines and Granzyme B were evaluated by Luminex assay using a custom MILLIPLEX® Human Cytokine/Chemokine Kit (Millipore, SRP1885), including the analytes of IFNγ, granzyme B, TNFα, and IL-12p70, as per manufacturer instructions. Serial dilutions of analyte standards were run in replicates on each assay plate. The Luminex plate was read on a FLEXMAP 3D© instrument (XMAP® technologies). Data was exported by XPONENT® Software and analyzed directly by EMD Millipore's MILLIPLEX® Analyst software, generating standard curves using a 5-parameter logistic non-linear regression fitting curve. The limits of detection (Min and Max) were calculated by the MILLIPLEX® Analyst software as the result of the average of appropriate replicate standard curve values obtained from each assay plate and indicate the range within which an analyte can be interpolated from the standards. Samples were run at appropriate dilutions to ensure measurements of sample analyte levels were within assay standard curve limits. Cytokine and granzyme B levels are reported in pg/mL or as fold-differences over IL12-RFP T cells (controls) and graphed in GraphPad Prism software.

TABLE 5
BLCLs for alloreactivity screen

| Cell Line Name | IHW Reference | Vendor |
|---|---|---|
| 1346-8357 | IHW01080 | Fred Hutch |
| 1347-8440 | IHW01103 | Fred Hutch |
| 1347-8442 | IHW01105 | Fred Hutch |
| 1416-1189 | IHW01176 | Fred Hutch |
| 1416-1337 | IHW01185 | Fred Hutch |
| FH19 | IHW09400 | Fred Hutch |
| FH31 | IHW09413 | Fred Hutch |
| FH39 | IHW09427 | Fred Hutch |
| FH46 | IHW09434 | Fred Hutch |
| FH70EY | IHW09458 | Fred Hutch |
| LCK | IHW09367 | Fred Hutch |
| TEM | IHW09057 | Fred Hutch |
| 165 | — | Astarte Biologics |
| FH18 | IHW09398 | Fred Hutch |
| FH21 | IHW09403 | Fred Hutch |
| FH25 | IHW09407 | Fred Hutch |
| FH3 | IHW09375 | Fred Hutch |
| FH36 | IHW09423 | Fred Hutch |
| FH43 | IHW09431 | Fred Hutch |
| FH53 | IHW09441 | Fred Hutch |
| FH6 | IHW09380 | Fred Hutch |
| FH9 | IHW09383 | Fred Hutch |
| ISH4 | IHW09371 | Fred Hutch |
| KT14 | IHW09103 | Fred Hutch |
| MYE 2003 | IHW09419 | Fred Hutch |
| MYE 2004 | IHW09420 | Fred Hutch |
| MYE 2006 | IHW09422 | Fred Hutch |
| SCL-116A | IHW09465 | Fred Hutch |
| T7526 | IHW09076 | Fred Hutch |
| TER-259 | IHW09401 | Fred Hutch |
| TUBO | IHW09045 | Fred Hutch |
| RSH | IHW09021 | Fred Hutch |
| WUZH1 | IHW09459 | Fred Hutch |
| 1333-8276 | IHW01040 | Fred Hutch |

### Expression and Purification of Protein Samples

sTCR Cloning, Expression and Purification

TCR chains were cloned into the microbial expression vector pFT28 by Golden Gate assembly The resulting plasmid containing either X6-31 TCRα or TCRβ in Boulter format (Boulter JM, Glick M, Todorov P T, et al. Protein Eng. 2003; 16(9):707-711.) was expressed separately as inclusion body in BL21 Star™ (DE3) by IPTG induction at 37° C. Detergent lysed and washed inclusion bodies (DWIBs) were prepared by cell lysis buffer (50 mM TRIS HCl, pH 8.0, 2 mM MgSO4 and 0.05 U/ml Benzonase, 1% Triton X-100 (v/v), 0.5% CHAPS (w/v)) and IB washing buffer (50 mM TRIS HCl pH8.0, 0.5% deoxy cholate, 2 mM EDTA) before storage at −80° C.

1 g of DWIB was solubilized in 7 ml of 140 mM Tris HCl pH 8.0, 6M GnHCl, 10 mM DTT and shaken at 30° C. for 2 h. Total protein concentration of solubilized DWIB was determined by Bradford assay to calculate the volume of solubilized DWIBs to have a molar ratio of 1:1 of each TCR α and β chain. Refolding by rapid dilution was performed by mixing of 50 mg solubilized DWIBs dropwise into 500 ml of refolding buffer containing 100 mM TRIS HCl pH 8.0, 40 mM Arginine (0.64 M stock in dH₂O), 2 mM EDTA, 5M Urea, final pH˜8.5, adding fresh 1.25 mM reduced glutathione (GSH), 6.6 mM reduced cysteine, 3.7 mM Cystamine (oxidized cysteamine). Refolded sTCR was dialyzed against 50 mM TRIS HCl pH 8.5, 1 mM EDTA in 20 kDa cutoff dialysis device for another 1˜2 days 4° C.

After filtration sTCR was loaded onto 10 ml Hitrap Q column and enriched via a linear NaCl gradient from 0 to 500 mM in 50 mM TRIS HCl pH 8.5, 1 mM EDTA. It was then further purified by gel filtration on a HiLoad 16/600 Superdex 75 pg column equilibrated with 30 mM HEPES pH 7.6, 150 mM NaCl. The peak fractions were pooled, concentrated and confirmed by SDS-PAGE and LC-MS.

Refolding of pMHC

Plasmids encoding huHLA and huB2M were separately expressed from BL21 cells. Inclusion bodies were isolated, washed with detergent and solubilized in 25 mM MES pH 6.0, 8 M urea, 10 mM EDTA, 0.1 mM DTT. Refolding reaction was carried out in the presence of 1000 nmole of B2M, 500 nmole of HLA and 15 mg of desired peptide in 100 mM Tris pH 8, 400 mM arginine HCl salt, 2 mM EDTA, 5 mM reduced glutathione, 0.5 mM oxidized glutathione and 0.2 mM PMSF to a total volume of 500 ml. HLA at 500 nmole was subsequently added twice and refolding reaction was allowed to proceed for up to four days at 4C. The refolding mixture was subsequently concentrated to about 10 ml and precipitates were removed by a 10-min spin using a benchtop centrifuge. It was buffer exchanged to size exclusion buffer such as 1×HBS (30 mM HEPES pH 7.6, 150 mM NaCl) and precipitates were again removed by spinning. The protein solution was then injected onto a HiLoad 26/600 Superdex 200 pg Cytiva with 1×HBS as the mobile phase. pMHC peak was pooled and frozen in liquid nitrogen.

TCR2 α-Chain-Va, huTRAC(1-94,T48C)
(SEQ ID NO: 78)
MAEDQVTQSPEALRLQEGESSSLNCSYTVSGLRGLFWYRQDPGKGPEFL
FTLYSAGEEKEKERLKATLTKKESFLHITAPKPEDSATYLCAVQAWGGG
SQGNLIFGKGTKLSVKPNIQNPDPAVYQLRDSKSSDKSVCLFTDFDSQT
NVSQSKDSDVYITDKCVLDMRSMDFKSNSAVAWSNKSDFACANAFNNSI
IPEDTFFPSPESS
TCR2 β-Chain-Vb, huTRBC2(1-130,S57C,C75A), GS,
Avitag
(SEQ ID NO: 79)
MADSGVTQTPKHLITATGQRVTLRCSPRSGDLSVYWYQQSLDQGLQFLI
QYYNGEERAKGNILERFSAQQFPDLHSELNLSSLELGDSALYFCASSAG
DRGYEQYFGPGTRLTVTEDLKNVFPPEVAVFEPSEAEISHTQKATLVCL
ATGFYPDHVELSWWVNGKEVHSGVCTDPQPLKEQPALNDSRYALSSRLR
VSATFWQNPRNHFRCQVQFYGLSENDEWTQDRAKPVTQIVSAEAWGRAD
GSLNDIFEAQKIEWHE
HLA-A2 (25-295)-LRWE
(SEQ ID NO: 80)
GSHSMRYFFTSVSRPGRGEPRFIAVGYVDDTQFVRFDSDAASQRMEPRA
PWIEQEGPEYWDGETRKVKAHSQTHRVDLGTLRGYYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLORTDAPKTHMTHHAVSD
HEATLRCWALSFYPAEITLTWORDGEDQTQDTELVETRPAGDGTFQKWA
AVVVPSGQEQRYTCHVQHEGLPKPLTLRWE
β2 (21-119)
(SEQ ID NO: 81)
MIQRTPKIQVYSRHPAENGKSNFLNCYVSGFHPSDIEVDLLKNGERIEK
VEHSDLSFSKDWSFYLLYYTEFTPTEKDEYACRVNHVTLSQPKIVKWDR
DM
DCAF4L2 peptide
(SEQ ID NO: 1)
ILQDGQFLV

### Complex Formation and Crystallization

The DCAF4L2 pMHC/TCR2 complex was made by mixing a molar excess of DCAF4L2 pMHC with TCR2. The complex was separated from excess DCAF4L2 pMHC by purification on a size exclusion chromatography column. The DCAF4L2 pMHC/TCR2 complex was concentrated to 12 mg/ml and crystallizes in 16% PEG 3350, 0.1M sodium citrate pH 5.6, 2% tacsimate pH 5.0, 10 mM calcium chloride.

### Data Collection and Structure Determination

The dataset for the DCAF4L2 pMHC/TCR2 complex crystal was collected on beamline 5.0.2 at the Berkeley synchrotron and processed with Mosfim/Aimless (Battye, et al., iMOSFLM: a new graphical interface for diffraction-image processing with MOSFLM. Acta Crystallogr D Biol Crystallogr 67, 271-81 (2011); Acta Crystallogr D Biol Crystallogr 50, 760-3 (1994)).

A previously solved low-resolutionhuman DCAF4L2 pMHC/TCR2 structure was used as a search model for the entire complex. The first in-house pMHC/sTCR structure was solved using a MHC structure (PDB code: 2PYE) as a search model for the MHC, and a sTCR structure (PDB code: 2PYE) as a search model for the TCR molecule.

DCAF4L2 pMHC/TCR2 complex crystals grow in the P213 space group with unit cell dimensions a=205.24, b=205.24, c=205.24 Å with one complex molecule per asymmetric unit and diffract to 2.8 Å resolution. The DCAF4L2 pMHC/X1-031 TCR-T complex structure was solved by molecular replacement with the program PHASER (Acta Crystallogr D Biol Crystallogr 50, 760-3 (1994)). The structure was improved with multiple rounds of model building with Coot (Emsley, et al., Features and development of Coot. Acta Crystallogr D Biol Crystallogr 66, 486-501 (2010) and refinement with Refmac, (Acta Crystallogr D Biol Crystallogr 50, 760-3 (1994)) to a final R=23.3/R_{free}=27.8)).

One full complex molecule, made up of Chains A, B, C, D and E was used for analysis. Core interaction interface amino acids were determined as being all amino acid residues with at least one non-hydrogen atom less than or equal to 5 Å from the partner protein. Amino acids that met these distance criteria were calculated with the program PyMOL (DeLano, The PyMOL Molecular Graphics System. (Palo Alto, 2002). FIGS. 13 and 14 show the crystal structure and interaction identification of the DCAF4L2 pMHC/TCR2 complex.

1. An expression vector comprising a nucleic acid sequence encoding a T-cell receptor (TCR) alpha chain and a TCR beta chain, wherein the TCR alpha chain and TCR beta chain are selected from the group consisting of:
a. a TCR alpha chain comprising an amino acid sequence set forth in SEQ ID NO:12 and a TCR beta chain comprising an amino acid sequence set forth in SEQ ID NO:23;
b. a TCR alpha chain comprising an amino acid sequence set forth in SEQ ID NO:13 and a TCR beta chain comprising an amino acid sequence set forth in SEQ ID NO:24;
c. a TCR alpha chain comprising an amino acid sequence set forth in SEQ ID NO:14 and a TCR beta chain comprising an amino acid sequence set forth in SEQ ID NO:25;
d. a TCR alpha chain comprising an amino acid sequence set forth in SEQ ID NO:15 and a TCR beta chain comprising an amino acid sequence set forth in SEQ ID NO:26;
e. a TCR alpha chain comprising an amino acid sequence set forth in SEQ ID NO:16 and a TCR beta chain comprising an amino acid sequence set forth in SEQ ID NO:27;
f. a TCR alpha chain comprising an amino acid sequence set forth in SEQ ID NO:17 and a TCR beta chain comprising an amino acid sequence set forth in SEQ ID NO:28;
g. a TCR alpha chain comprising an amino acid sequence set forth in SEQ ID NO: 18 and a TCR beta chain comprising an amino acid sequence set forth in SEQ ID NO:29;
h. a TCR alpha chain comprising an amino acid sequence set forth in SEQ ID NO:19 and a TCR beta chain comprising an amino acid sequence set forth in SEQ ID NO:30;
i. a TCR alpha chain comprising an amino acid sequence set forth in SEQ ID NO:20 and a TCR beta chain comprising an amino acid sequence set forth in SEQ ID NO:31;
j. a TCR alpha chain comprising an amino acid sequence set forth in SEQ ID NO:21 and a TCR beta chain comprising an amino acid sequence set forth in SEQ ID NO:32; and
k. a TCR alpha chain comprising an amino acid sequence set forth in SEQ ID NO:22 and a TCR beta chain comprising an amino acid sequence set forth in SEQ ID NO:33.

2. The expression vector of claim 1, further comprising a nucleic acid encoding interleukin-12 (IL-12) or a functional variant thereof.

3. The expression vector of claim 1, wherein the expression vector is a viral vector.

4. The expression vector of claim 3, wherein the viral vector is selected from a retroviral vector or a lentiviral vector.

5. The expression vector of claim 4, wherein the retroviral vector is a lentiviral vector.

6. The expression vector of claim 1, wherein the TCR alpha chain comprises an amino acid sequence set forth in SEQ ID NO: 12 and the TCR beta chain comprises an amino acid sequence set forth in SEQ ID NO:23.

7. The expression vector of claim 6, wherein the TCR alpha chain comprises an amino acid sequence set forth in SEQ ID NO:34 and the TCR beta chain comprises an amino acid sequence set forth in SEQ ID NO:45.

8. The expression vector of claim 7, wherein the TCR alpha chain comprises an amino acid sequence set forth in SEQ ID NO:56 and the TCR beta chain comprises an amino acid sequence set forth in SEQ ID NO:67.

9. The expression vector of claim 1, wherein the TCR alpha chain comprises an amino acid sequence set forth in SEQ ID NO:13 and the TCR beta chain comprises an amino acid sequence set forth in SEQ ID NO:24.

10. The expression vector of claim 9, wherein the TCR alpha chain comprises an amino acid sequence set forth in SEQ ID NO:35 and the TCR beta chain comprises an amino acid sequence set forth in SEQ ID NO:46.

11. The expression vector of claim 10, wherein the TCR alpha chain comprises an amino acid sequence set forth in SEQ ID NO:57 and the TCR beta chain comprises an amino acid sequence set forth in SEQ ID NO:68.

12. The expression vector of claim 1, wherein the TCR alpha chain comprises an amino acid sequence set forth in SEQ ID NO:14 and the TCR beta chain comprises an amino acid sequence set forth in SEQ ID NO:25.

13. The expression vector of claim 12, wherein the TCR alpha chain comprises an amino acid sequence set forth in SEQ ID NO:36 and the TCR beta chain comprises an amino acid sequence set forth in SEQ ID NO:47.

14. The expression vector of claim 13, wherein the TCR alpha chain comprises an amino acid sequence set forth in SEQ ID NO:58 and the TCR beta chain comprises an amino acid sequence set forth in SEQ ID NO:69.

15. The expression vector of claim 1, wherein the TCR alpha chain comprises an amino acid sequence set forth in SEQ ID NO:15 and the TCR beta chain comprises an amino acid sequence set forth in SEQ ID NO:26.

16. The expression vector of claim 15, wherein the TCR alpha chain comprises an amino acid sequence set forth in SEQ ID NO:37 and the TCR beta chain comprises an amino acid sequence set forth in SEQ ID NO:48.

17. The expression vector of claim 16, wherein the TCR alpha chain comprises an amino acid sequence set forth in SEQ ID NO:59 and the TCR beta chain comprises an amino acid sequence set forth in SEQ ID NO:70.

18. The expression vector of any of claim 1, wherein the TCR alpha chain comprises an amino acid sequence set forth in SEQ ID NO:16 and the TCR beta chain comprises an amino acid sequence set forth in SEQ ID NO:27.

19. The expression vector of claim 18, wherein the TCR alpha chain comprises an amino acid sequence set forth in SEQ ID NO:38 and the TCR beta chain comprises an amino acid sequence set forth in SEQ ID NO:40.

20. The expression vector of claim 19, wherein the TCR alpha chain comprises an amino acid sequence set forth in SEQ ID NO:60 and the TCR beta chain comprises an amino acid sequence set forth in SEQ ID NO:71.

21. The expression vector of claim 1, wherein the TCR alpha chain comprises an amino acid sequence set forth in SEQ ID NO:17 and the TCR beta chain comprises an amino acid sequence set forth in SEQ ID NO:28.

22. The expression vector of claim 21, wherein the TCR alpha chain comprises an amino acid sequence set forth in SEQ ID NO:39 and the TCR beta chain comprises an amino acid sequence set forth in SEQ ID NO:50.

23. The expression vector of claim 21, wherein the TCR alpha chain comprises an amino acid sequence set forth in SEQ ID NO:61 and the TCR beta chain comprises an amino acid sequence set forth in SEQ ID NO:72.

24. The expression vector of claim 1, wherein the TCR alpha chain comprises an amino acid sequence set forth in SEQ ID NO:18 and the TCR beta chain comprises an amino acid sequence set forth in SEQ ID NO:29.

25. The expression vector of claim 24, wherein the TCR alpha chain comprises an amino acid sequence set forth in SEQ ID NO:40 and the TCR beta chain comprises an amino acid sequence set forth in SEQ ID NO:51.

26. The expression vector of claim 25, wherein the TCR alpha chain comprises an amino acid sequence set forth in SEQ ID NO:62 and the TCR beta chain comprises an amino acid sequence set forth in SEQ ID NO: 73.

27. The expression vector of claim 1, wherein the TCR alpha chain comprises an amino acid sequence set forth in SEQ ID NO:19 and the TCR beta chain comprises an amino acid sequence set forth in SEQ ID NO:30.

28. The expression vector of claim 27, wherein the TCR alpha chain comprises an amino acid sequence set forth in SEQ ID NO:41 and the TCR beta chain comprises an amino acid sequence set forth in SEQ ID NO:52.

29. The expression vector of claim 28, wherein the TCR alpha chain comprises an amino acid sequence set forth in SEQ ID NO:63 and the TCR beta chain comprises an amino acid sequence set forth in SEQ ID NO:74.

30. The expression vector of claim 1, wherein the TCR alpha chain comprises an amino acid sequence set forth in SEQ ID NO:20 and the TCR beta chain comprises an amino acid sequence set forth in SEQ ID NO:31.

31. The expression vector of claim 30, wherein the TCR alpha chain comprises an amino acid sequence set forth in SEQ ID NO:42 and the TCR beta chain comprises an amino acid sequence set forth in SEQ ID NO:53.

32. The expression vector of claim 31, wherein the TCR alpha chain comprises an amino acid sequence set forth in SEQ ID NO:64 and the TCR beta chain comprises an amino acid sequence set forth in SEQ ID NO:75.

33. The expression vector of claim 1, wherein the TCR alpha chain comprises an amino acid sequence set forth in SEQ ID NO:21 and the TCR beta chain comprises an amino acid sequence set forth in SEQ ID NO:32.

34. The expression vector of claim 33, wherein the TCR alpha chain comprises an amino acid sequence set forth in SEQ ID NO:43 and the TCR beta chain comprises an amino acid sequence set forth in SEQ ID NO:54.

35. The expression vector of claim 34, wherein the TCR alpha chain comprises an amino acid sequence set forth in SEQ ID NO:65 and the TCR beta chain comprises an amino acid sequence set forth in SEQ ID NO:76.

36. The expression vector of claim 1, wherein the TCR alpha chain comprises an amino acid sequence set forth in SEQ ID NO:22 and the TCR beta chain comprises an amino acid sequence set forth in SEQ ID NO:33.

37. The expression vector of claim 36, wherein the TCR alpha chain comprises an amino acid sequence set forth in SEQ ID NO:44 and the TCR beta chain comprises an amino acid sequence set forth in SEQ ID NO:55.

38. The expression vector of claim 37, wherein the TCR alpha chain comprises an amino acid sequence set forth in SEQ ID NO:66 and the TCR beta chain comprises an amino acid sequence set forth in SEQ ID NO:77.

39. A cell expressing a recombinant T-cell receptor (TCR), said TCR comprising:
a. a TCR alpha chain comprising an amino acid sequence set forth in SEQ ID NO:12 and a TCR beta chain comprising an amino acid sequence set forth in SEQ ID NO:23;
b. a TCR alpha chain comprising an amino acid sequence set forth in SEQ ID NO:13 and a TCR beta chain comprising an amino acid sequence set forth in SEQ ID NO:24;
c. a TCR alpha chain comprising an amino acid sequence set forth in SEQ ID NO:14 and a TCR beta chain comprising an amino acid sequence set forth in SEQ ID NO:25;
d. a TCR alpha chain comprising an amino acid sequence set forth in SEQ ID NO:15 and a TCR beta chain comprising an amino acid sequence set forth in SEQ ID NO:26;
e. a TCR alpha chain comprising an amino acid sequence set forth in SEQ ID NO:16 and a TCR beta chain comprising an amino acid sequence set forth in SEQ ID NO:27;
f. a TCR alpha chain comprising an amino acid sequence set forth in SEQ ID NO:17 and a TCR beta chain comprising an amino acid sequence set forth in SEQ ID NO:28;
g. a TCR alpha chain comprising an amino acid sequence set forth in SEQ ID NO: 18 and a TCR beta chain comprising an amino acid sequence set forth in SEQ ID NO:29;
h. a TCR alpha chain comprising an amino acid sequence set forth in SEQ ID NO:19 and a TCR beta chain comprising an amino acid sequence set forth in SEQ ID NO:30;
i. a TCR alpha chain comprising an amino acid sequence set forth in SEQ ID NO:20 and a TCR beta chain comprising an amino acid sequence set forth in SEQ ID NO:31;
j. a TCR alpha chain comprising an amino acid sequence set forth in SEQ ID NO:21 and a TCR beta chain comprising an amino acid sequence set forth in SEQ ID NO:32; or
k. a TCR alpha chain comprising an amino acid sequence set forth in SEQ ID NO:22 and a TCR beta chain comprising an amino acid sequence set forth in SEQ ID NO:33.

40. The cell of claim 39, said TCR comprising:
a. a TCR alpha chain comprising an amino acid sequence set forth in SEQ ID NO:34 and a TCR beta chain comprising an amino acid sequence set forth in SEQ ID NO:45;
b. a TCR alpha chain comprising an amino acid sequence set forth in SEQ ID NO:35 and a TCR beta chain comprising an amino acid sequence set forth in SEQ ID NO:46;
c. a TCR alpha chain comprising an amino acid sequence set forth in SEQ ID NO:36 and a TCR beta chain comprising an amino acid sequence set forth in SEQ ID NO:47;
d. a TCR alpha chain comprising an amino acid sequence set forth in SEQ ID NO:37 and a TCR beta chain comprising an amino acid sequence set forth in SEQ ID NO:48;
e. a TCR alpha chain comprising an amino acid sequence set forth in SEQ ID NO:38 and a TCR beta chain comprising an amino acid sequence set forth in SEQ ID NO:49;
f. a TCR alpha chain comprising an amino acid sequence set forth in SEQ ID NO:39 and a TCR beta chain comprising an amino acid sequence set forth in SEQ ID NO:50;
g. a TCR alpha chain comprising an amino acid sequence set forth in SEQ ID NO:40 and a TCR beta chain comprising an amino acid sequence set forth in SEQ ID NO:51;
h. a TCR alpha chain comprising an amino acid sequence set forth in SEQ ID NO:41 and a TCR beta chain comprising an amino acid sequence set forth in SEQ ID NO:52;
i. a TCR alpha chain comprising an amino acid sequence set forth in SEQ ID NO:42 and a TCR beta chain comprising an amino acid sequence set forth in SEQ ID NO:53;
j. a TCR alpha chain comprising an amino acid sequence set forth in SEQ ID NO:43 and a TCR beta chain comprising an amino acid sequence set forth in SEQ ID NO:54; or
k. a TCR alpha chain comprising an amino acid sequence set forth in SEQ ID NO:44 and a TCR beta chain comprising an amino acid sequence set forth in SEQ ID NO:55.

41. The cell of claim 39, wherein the cell further expresses a recombinant IL-12 or functional variant thereof.

42. A cell comprising an expression vector of claim 1.

43. The cell of claim 39, wherein the cell is a T cell.

44. The cell of claim 43, wherein the TCR binds the peptide of SEQ ID NO:1 or SEQ ID NO:2 in the context of HLA-A*02:01 and said binding leads to activation of IFNγ, TNFα, or granzyme B production by said cell.

45. A pharmaceutical composition comprising a therapeutically effect amount of a cell of claim 39.

46. A method of making a cell of claim 39, comprising introducing into a cell an expression vector comprising a nucleic acid sequence encoding a TCR alpha chain and a TCR beta chain, wherein the TCR alpha chain and TCR beta chain are selected from the group consisting of:
a. a TCR alpha chain comprising an amino acid sequence set forth in SEQ ID NO:12 and a TCR beta chain comprising an amino acid sequence set forth in SEQ ID NO:23;
b. a TCR alpha chain comprising an amino acid sequence set forth in SEQ ID NO:13 and a TCR beta chain comprising an amino acid sequence set forth in SEQ ID NO:24;
c. a TCR alpha chain comprising an amino acid sequence set forth in SEQ ID NO:14 and a TCR beta chain comprising an amino acid sequence set forth in SEQ ID NO:25;
d. a TCR alpha chain comprising an amino acid sequence set forth in SEQ ID NO:15 and a TCR beta chain comprising an amino acid sequence set forth in SEQ ID NO:26;
e. a TCR alpha chain comprising an amino acid sequence set forth in SEQ ID NO:16 and a TCR beta chain comprising an amino acid sequence set forth in SEQ ID NO:27;
f. a TCR alpha chain comprising an amino acid sequence set forth in SEQ ID NO:17 and a TCR beta chain comprising an amino acid sequence set forth in SEQ ID NO:28;
g. a TCR alpha chain comprising an amino acid sequence set forth in SEQ ID NO: 18 and a TCR beta chain comprising an amino acid sequence set forth in SEQ ID NO:29;
h. a TCR alpha chain comprising an amino acid sequence set forth in SEQ ID NO:19 and a TCR beta chain comprising an amino acid sequence set forth in SEQ ID NO:30;
i. a TCR alpha chain comprising an amino acid sequence set forth in SEQ ID NO:20 and a TCR beta chain comprising an amino acid sequence set forth in SEQ ID NO:31;
j. a TCR alpha chain comprising an amino acid sequence set forth in SEQ ID NO:21 and a TCR beta chain comprising an amino acid sequence set forth in SEQ ID NO:32; or
k. a TCR alpha chain comprising an amino acid sequence set forth in SEQ ID NO:22 and a TCR beta chain comprising an amino acid sequence set forth in SEQ ID NO:33.

47. The method of claim 46, wherein the TCR alpha chain and TCR beta chain are selected from the group consisting of:
a. a TCR alpha chain comprising an amino acid sequence set forth in SEQ ID NO:34 and a TCR beta chain comprising an amino acid sequence set forth in SEQ ID NO:45;
b. a TCR alpha chain comprising an amino acid sequence set forth in SEQ ID NO:35 and a TCR beta chain comprising an amino acid sequence set forth in SEQ ID NO:46;
c. a TCR alpha chain comprising an amino acid sequence set forth in SEQ ID NO:36 and a TCR beta chain comprising an amino acid sequence set forth in SEQ ID NO:47;
d. a TCR alpha chain comprising an amino acid sequence set forth in SEQ ID NO:37 and a TCR beta chain comprising an amino acid sequence set forth in SEQ ID NO:48;
e. a TCR alpha chain comprising an amino acid sequence set forth in SEQ ID NO:38 and a TCR beta chain comprising an amino acid sequence set forth in SEQ ID NO:49;
f. a TCR alpha chain comprising an amino acid sequence set forth in SEQ ID NO:39 and a TCR beta chain comprising an amino acid sequence set forth in SEQ ID NO:50;
g. a TCR alpha chain comprising an amino acid sequence set forth in SEQ ID NO:40 and a TCR beta chain comprising an amino acid sequence set forth in SEQ ID NO:51;
h. a TCR alpha chain comprising an amino acid sequence set forth in SEQ ID NO:41 and a TCR beta chain comprising an amino acid sequence set forth in SEQ ID NO: 52;
i. a TCR alpha chain comprising an amino acid sequence set forth in SEQ ID NO:43 and a TCR beta chain comprising an amino acid sequence set forth in SEQ ID NO:54;
j. a TCR alpha chain comprising an amino acid sequence set forth in SEQ ID NO:43 and a TCR beta chain comprising an amino acid sequence set forth in SEQ ID NO:54; and
k. a TCR alpha chain comprising an amino acid sequence set forth in SEQ ID NO:44 and a TCR beta chain comprising an amino acid sequence set forth in SEQ ID NO:55.

48. The method of claim 46, wherein the expression vector further comprises a nucleic acid sequence encoding IL-12 or a functional variant thereof.

49. The method of claim 46, wherein the cell is a T cell.

50. The method of claim 49, wherein the T cell is a primary T cell.

51. The method of 50, wherein the primary T cell is isolated from a cancer patient.

52. A method of treating a DCAF4L2 expressing cancer, said method comprising administering to a cancer patient a therapeutically effective amount of a cell of of claim 39.

53. The method of claim 52, wherein the patient is tested prior to administration to determine the presence of a cancer expressing DCAF4L2.

54. The method of claim 53, wherein a nucleic acid encoding DCAF4L2 is detected.

55. The method of claim 53, wherein DCAF4L2 protein or a DCAF4L2-derived peptide is detected.

56. The method of claim 52, wherein the patient is identified to carry the HLA-A*02:01 allele.

57. A method of making a pharmaceutical composition of claim 45, comprising introducing into a cell an expression vector comprising a nucleic acid sequence encoding a TCR alpha chain and a TCR beta chain, wherein the TCR alpha chain and TCR beta chain are selected from the group consisting of:
a. a TCR alpha chain comprising an amino acid sequence set forth in SEQ ID NO:12 and a TCR beta chain comprising an amino acid sequence set forth in SEQ ID NO:23;
b. a TCR alpha chain comprising an amino acid sequence set forth in SEQ ID NO:13 and a TCR beta chain comprising an amino acid sequence set forth in SEQ ID NO:24;
c. a TCR alpha chain comprising an amino acid sequence set forth in SEQ ID NO:14 and a TCR beta chain comprising an amino acid sequence set forth in SEQ ID NO:25;
d. a TCR alpha chain comprising an amino acid sequence set forth in SEQ ID NO:15 and a TCR beta chain comprising an amino acid sequence set forth in SEQ ID NO:26;
e. a TCR alpha chain comprising an amino acid sequence set forth in SEQ ID NO:16 and a TCR beta chain comprising an amino acid sequence set forth in SEQ ID NO:27;
f. a TCR alpha chain comprising an amino acid sequence set forth in SEQ ID NO:17 and a TCR beta chain comprising an amino acid sequence set forth in SEQ ID NO:28;
g. a TCR alpha chain comprising an amino acid sequence set forth in SEQ ID NO: 18 and a TCR beta chain comprising an amino acid sequence set forth in SEQ ID NO:29;
h. a TCR alpha chain comprising an amino acid sequence set forth in SEQ ID NO:19 and a TCR beta chain comprising an amino acid sequence set forth in SEQ ID NO:30;
i. a TCR alpha chain comprising an amino acid sequence set forth in SEQ ID NO:20 and a TCR beta chain comprising an amino acid sequence set forth in SEQ ID NO:31;
j. a TCR alpha chain comprising an amino acid sequence set forth in SEQ ID NO:21 and a TCR beta chain comprising an amino acid sequence set forth in SEQ ID NO:32; or
k. a TCR alpha chain comprising an amino acid sequence set forth in SEQ ID NO:22 and a TCR beta chain comprising an amino acid sequence set forth in SEQ ID NO:33.

58. The method of claim 57, wherein the TCR alpha chain and TCR beta chain are selected from the group consisting of:
a. a TCR alpha chain comprising an amino acid sequence set forth in SEQ ID NO:34 and a TCR beta chain comprising an amino acid sequence set forth in SEQ ID NO:45;
b. a TCR alpha chain comprising an amino acid sequence set forth in SEQ ID NO:35 and a TCR beta chain comprising an amino acid sequence set forth in SEQ ID NO:46;
c. a TCR alpha chain comprising an amino acid sequence set forth in SEQ ID NO:36 and a TCR beta chain comprising an amino acid sequence set forth in SEQ ID NO:47;
d. a TCR alpha chain comprising an amino acid sequence set forth in SEQ ID NO:37 and a TCR beta chain comprising an amino acid sequence set forth in SEQ ID NO:48;
e. a TCR alpha chain comprising an amino acid sequence set forth in SEQ ID NO:38 and a TCR beta chain comprising an amino acid sequence set forth in SEQ ID NO:49;
f. a TCR alpha chain comprising an amino acid sequence set forth in SEQ ID NO:39 and a TCR beta chain comprising an amino acid sequence set forth in SEQ ID NO:50;
g. a TCR alpha chain comprising an amino acid sequence set forth in SEQ ID NO:40 and a TCR beta chain comprising an amino acid sequence set forth in SEQ ID NO:51;
h. a TCR alpha chain comprising an amino acid sequence set forth in SEQ ID NO:41 and a TCR beta chain comprising an amino acid sequence set forth in SEQ ID NO: 52;
i. a TCR alpha chain comprising an amino acid sequence set forth in SEQ ID NO:43 and a TCR beta chain comprising an amino acid sequence set forth in SEQ ID NO: 54;
j. a TCR alpha chain comprising an amino acid sequence set forth in SEQ ID NO:43 and a TCR beta chain comprising an amino acid sequence set forth in SEQ ID NO:54; and
k. a TCR alpha chain comprising an amino acid sequence set forth in SEQ ID NO:44 and a TCR beta chain comprising an amino acid sequence set forth in SEQ ID NO:55.

59. A method of treating a DCAF4L2 expressing cancer, said method comprising administering to a cancer patient a therapeutically effective amount of a pharmaceutical composition of claim 45.

60. A method of treating a DCAF4L2 expressing cancer, said method comprising administering to a cancer patient a therapeutically effective amount of a cell made by the method of claim 46.


SEQUENCE LISTING (81 sequences)

SEQ ID NO: 1 - protein, 9 aa, Homo sapiens
ILQDGQFLV

SEQ ID NO: 2 - protein, 9 aa, Homo sapiens
ILQDEQYLM

SEQ ID NO: 3 - protein, 9 aa, Homo sapiens
TLTDEQFLV

SEQ ID NO: 4 - protein, 9 aa, Homo sapiens
GLVDGLFLV

SEQ ID NO: 5 - protein, 9 aa, Homo sapiens
ILQEGTFKA

SEQ ID NO: 6 - protein, 9 aa, Homo sapiens
ILQDGRIFI

SEQ ID NO: 7 - protein, 9 aa, Homo sapiens
LLQDKQFEL

SEQ ID NO: 8 - protein, 9 aa, Homo sapiens
ILTDVTLLV

SEQ ID NO: 9 - protein, 9 aa, Homo sapiens
ILLDNEFHV

SEQ ID NO: 10 - protein, 9 aa, Homo sapiens
ILFESQFSV

SEQ ID NO: 11 - protein, 9 aa, Homo sapiens
TLQNSQFLL

SEQ ID NO: 12 - protein, 12 aa, Homo sapiens
CVLKGGGNKLTF

SEQ ID NO: 13 - protein, 16 aa, Homo sapiens
CAVQAWGGGSQGNLIF

SEQ ID NO: 14 - protein, 17 aa, Homo sapiens
CVVNGINRGSTLGRLYF

SEQ ID NO: 15 - protein, 11 aa, Homo sapiens
CVVSAYARLMF

SEQ ID NO: 16 - protein, 16 aa, Homo sapiens
CGADELGIQGAQKLVF

SEQ ID NO: 17 - protein, 17 aa, Homo sapiens
CVVIEGNYGGATNKLIF

SEQ ID NO: 18 - protein, 16 aa, Homo sapiens
CAMREPYKYNNNDMRF

SEQ ID NO: 19 - protein, 14 aa, Homo sapiens
CAYPLVNQGGKLIF

SEQ ID NO: 20 - protein, 14 aa, Homo sapiens
CGALASGGYQKVTF

SEQ ID NO: 21 - protein, 14 aa, Homo sapiens
CAVSNMDSSYKLIF

SEQ ID NO: 22 - protein, 12 aa, Homo sapiens
CAVPTGNTPLVF

SEQ ID NO: 23 - protein, 15 aa, Homo sapiens
CASSYPTGAYYGYTF

SEQ ID NO: 24 - protein, 14 aa, Homo sapiens
CASSAGDRGYEQYF

SEQ ID NO: 25 - protein, 17 aa, Homo sapiens
CASSGGYWDQENTEAFF

SEQ ID NO: 26 - protein, 16 aa, Homo sapiens
CASSLGGQGGAGELFF

SEQ ID NO: 27 - protein, 15 aa, Homo sapiens
CASSKVAGAIETQYF

SEQ ID NO: 28 - protein, 13 aa, Homo sapiens
CASSKGIRYEQYF

SEQ ID NO: 29 - protein, 15 aa, Homo sapiens
CASSARQGTNEKLFF

SEQ ID NO: 30 - protein, 14 aa, Homo sapiens
CASSLVATWDEQFF

SEQ ID NO: 31 - protein, 17 aa, Homo sapiens
CASSPSGTPSGANVLTF

SEQ ID NO: 32 - protein, 17 aa, Homo sapiens
CSALPSGVDRLNTEAFF

SEQ ID NO: 33 - protein, 14 aa, Homo sapiens
CAATGASRPTEAFF

SEQ ID NO: 34 - protein, 253 aa, Homo sapiens
AQSVTQLDSHVSVSEGTPVLLRCNYSSSYSPSLFWYVQHPNKGLQLLLKYTSAATLVKGINGFEAEFKKSETSFHLTKPSAHMSDAAEYFCVLKGGGNKLTFGTGTQLKVELNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 35 - protein, 254 aa, Homo sapiens
EDQVTQSPEALRLQEGESSSLNCSYTVSGLRGLFWYRQDPGKGPEFLFTLYSAGEEKEKERLKATLTKKESFLHITAPKPEDSATYLCAVQAWGGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 255 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNGINRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 37 - protein, 251 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVVSAYARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 255 aa, Homo sapiens
SQELEQSPQSLIVQEGKNLTINCTSSKTLYGLYWYKQKYGEGLIFLMMLQKGGEEKSHEKITAKLDEKKQQSSLHITASQPSHAGIYLCGADELGIQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 255 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVIEGNYGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 258 aa, Homo sapiens
AQKITQTQPGMFVQEKEAVTLDCTYDTSDQSYGLFWYKQPSSGEMIFLIYQGSYDEQNATEGRYSLNFQKARKSANLVISASQLGDSAMYFCAMREPYKYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 41 - protein, 256 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYPLVNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 253 aa, Homo sapiens
SQELEQSPQSLIVQEGKNLTINCTSSKTLYGLYWYKQKYGEGLIFLMMLQKGGEEKSHEKITAKLDEKKQQSSLHITASQPSHAGIYLCGALASGGYQKVTFGIGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 43 - protein, 255 aa, Homo sapiens
AQSVTQLGSHVSVSEGALVLLRCNYSSSVPPYLFWYVQYPNQGLQLLLKYTSAATLVKGINGFEAEFKKSETSFHLTKPSAHMSDAAEYFCAVSNMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 251 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVPTGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 45 - protein, 291 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYPTGAYYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 46 - protein, 292 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSAGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 47 - protein, 293 aa, Homo sapiens
EADIYQTPRYLVIGTGKKITLECSQTMGHDKMYWYQQDPGMELHLIHYSYGVNSTEKGDLSSESTVSRIRTEHFPLTLESARPSHTSQYLCASSGGYWDQENTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 48 - protein, 295 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGGQGGAGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 49 - protein, 294 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSKVAGAIETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 50 - protein, 291 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSKGIRYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - protein, 291 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSARQGTNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 52 - protein, 293 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLVATWDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 53 - protein, 295 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPSGTPSGANVLTFGAGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 54 - protein, 296 aa, Homo sapiens
GAVVSQHPSRVICKSGTSVKIECRSLDFQATTMFWYRQFPKKSLMLMATSNEGSKATYEQGVEKDKFLINHASLTLSTLTVTSAHPEDSSFYICSALPSGVDRLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 55 - protein, 290 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCAATGASRPTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 56 - protein, 272 aa, Homo sapiens
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVLKGGGNKLTFGTGTQLKVELNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 57 - protein, 275 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQAWGGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 276 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNGINRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 59 - protein, 272 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAYARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 275 aa, Homo sapiens
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADELGIQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 61 - protein, 276 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVIEGNYGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 278 aa, Homo sapiens
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREPYKYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 63 - protein, 276 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYPLVNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 273 aa, Homo sapiens
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGALASGGYQKVTFGIGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - protein, 274 aa, Homo sapiens
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSNMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 272 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVPTGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 67 - protein, 310 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYPTGAYYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 68 - protein, 311 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 312 aa, Homo sapiens
MTIRLLCYVGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSGGYWDQENTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 70 - protein, 314 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGGQGGAGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 71 - protein, 313 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSKVAGAIETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 72 - protein, 310 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSKGIRYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 310 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSARQGTNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 74 - protein, 312 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLVATWDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 75 - protein, 314 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPSGTPSGANVLTFGAGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 76 - protein, 310 aa, Homo sapiens
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSALPSGVDRLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 77 - protein, 309 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCAATGASRPTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 78 - protein, 209 aa, Homo sapiens
MAEDQVTQSPEALRLQEGESSSLNCSYTVSGLRGLFWYRQDPGKGPEFLFTLYSAGEEKEKERLKATLTKKESFLHITAPKPEDSATYLCAVQAWGGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 79 - protein, 261 aa, Homo sapiens
MADSGVTQTPKHLITATGQRVTLRCSPRSGDLSVYWYQQSLDQGLQFLIQYYNGEERAKGNILERFSAQQFPDLHSELNLSSLELGDSALYFCASSAGDRGYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGSLNDIFEAQKIEWHE

SEQ ID NO: 80 - protein, 275 aa, Homo sapiens [sequence omitted]

SEQ ID NO: 81 - protein, 100 aa, Homo sapiens [sequence omitted]